(19) United States
(12) Patent Application Publication
(10) Pub. No.: US 2003/0190746 A1
(43) Pub. Date: Oct. 9, 2003

(54) Gene expression control system and its use in recombinant virus packaging cell lines

(76) Inventor: Xiao Xiao, Wexford, PA (US)

Correspondence Address: KIRKPATRICK & LOCKHART LLP, 535 SMITHFIELD STREET, PITTSBURGH, PA, 15222, US

(21) Appl. No.: 10/252,279

(22) Filed: Sep. 23, 2002

Related U.S. Application Data
(60) Provisional application No. 60/369,987, filed on Apr. 4, 2002.

(51) Int. Cl. A61K 39/12; C12N 15/00; C12N 15/09; C12N 15/63; C12N 15/70; C12N 15/74

(52) U.S. Cl. 435/320.1; 424/199.1

(57) ABSTRACT

Provided is a gene expression control method that employs a transcription termination sequence positioned within an intron. The transcription termination sequence is disruptable by the addition of a trans-acting factor. For example, in a “dual splicing switch,” the transcription termination sequence is flanked by recombination sites and can be excised by a recombinase. The Cre/LoxP recombination system may be used for this purpose. In use, the intron containing the disruptable transcription termination sequence is positioned within a reading frame of a target gene. Also provided is a nucleic acid containing this transcription control mechanism and a cell line harboring a gene containing the nucleic acid. In one example, the transcription control mechanism is used to produce a cell line containing a toxic gene, the AAV Rep gene. A cell line for producing recombinant AAV virus particles, or recombinant parvovirus particles, also is provided along with a method for producing the same.

### BACKGROUND

1. Field of the Invention

A method is provided for controlling gene expression, finding particular usefulness in establishing cell lines containing cytotoxic or cytostatic genes, cell lines for producing recombinant virus particles, such as recombinant Adeno-associated virus (rAAV) virus particles.

2. Description of the Related Art

The recombinant adeno-associated viral vector system is derived from non-pathogenic and defective parvoviruses. Adeno-associated virus (AAV) vectors have been successfully used to establish efficient and long-term gene transfer in vivo in a variety of tissues without significant cellular immune responses or toxicity. The success of preclinical studies has led to clinical trials using AAV vectors to treat genetic diseases such as cystic fibrosis, hemophilia, and muscular dystrophy (Flotte, T. R. and Laube, B. L. 2001. Gene therapy in cystic fibrosis. Chest 120:124S-131S; High, K. A. 2001. AAV-mediated gene transfer for hemophilia. Ann N Y Acad Sci 953:64-74; and Stedman, H., Wilson, J. M., Finke, R., Kleckner, A. L. and Mendell, J. 2000. Phase I clinical trial utilizing gene therapy for limb girdle muscular dystrophy: alpha-, beta-, gamma-, or delta-sarcoglycan gene delivered with intramuscular instillations of adeno-associated vectors. Hum Gene Ther 11:777-90). Although the use of AAV vectors for human gene therapy is promising, current vector production methods may not meet the demand for AAV vectors in clinical studies involving certain genetic diseases, particularly for ones that require large quantity and high quality vectors. Gene therapy for muscular dystrophy (Li, J., Dressman, D., Tsao, Y. P., Sakamoto, A., Hoffman, E. P. and Xiao, X. 1999. rAAV vector-mediated sarcogylcan gene transfer in a hamster model for limb girdle muscular dystrophy. Gene Ther 6:74-82; and Wang, B., Li, J. and Xiao, X. 2000. Adeno-associated virus vector carrying human minidystrophin genes effectively ameliorates muscular dystrophy in mdx mouse model. Proc Natl Acad Sci USA A 97:13714-9), for example, requires gene transfer to large groups of muscles and even muscles of the entire body. These treatments require an enormous amount of clinical grade vectors. The AAV vector system is also being used as a gene transfer tool for functional genomics research. High-yield and scalable production methods are needed to meet the demands for AAV vectors in clinical trials.

Of numerous approaches to improve AAV vector production, two different strategies are now widely used. One method is based on the adenovirus-free transient transfection of all elements (vector and packaging plasmids along with helper genes isolated from Ad), which are required for AAV production in host cells such as 293 cells (Grimm, D. and Kleinschmidt, J. A. 1999. Progress in adeno-associated virus type 2 vector production: promises and prospects for clinical use. Hum Gene Ther 10:2445-50; and Xiao, X., Li, J. and Samulski, R. J. 1998. Production of high-titer recombinant adeno-associated virus vectors in the absence of helper adenovirus. J Virol 72:2224-32). The other method relies on wild type adenovirus infection into the cell lines that stably harbor AAV Rep/Cap genes as well as the AAV vector DNA. Although the transient transfection method generates high-titer AAV vectors that are free of adenovirus, it is labor intensive and expensive to scale up for clinical studies. On the other hand, the wild type adenovirus-inducible AAV producer cell lines can be scaled up in cultures and can produce AAV vectors with titers comparable to the transient transfection method. However, this approach faces a problem of the traditional method, namely the production of wild type helper adenovirus. Contamination of wild type adenovirus is highly undesirable in view of vector safety. As a result, new packaging cell lines are needed for AAV production to meet the demand for high quality and high quantity gene vectors in both preclinical and clinical studies.

Previous packaging cell lines featuring both stability and high-productivity are almost exclusively derived from cells which do not harbor the adenovirus E1A/E1B genes, for instance, HeLa cells (Chadeuf, G., Favre, D., Tessier, J., Provost, N., Nony, P., Kleinschmidt, J., Moullier, P. and Salvetti, A. 2000. Efficient recombinant adeno-associated virus production by a stable rep-cap HeLa cell line correlates with adenovirus-induced amplification of the integrated rep-cap genome. J Gene Med 2:260-8; and Clark, K. R., Voulgaropoulou, F., Fraley, D. M. and Johnson, P. R. 1995. Cell lines for the production of recombinant adeno-associated virus. Hum Gene Ther 6:1329-41). Production of AAV vectors from those packaging cell lines requires the infection of wild type adenovirus. On the other hand, human 293 cells, the most commonly used cells for AAV vector production by the transient transfection method, contain adenovirus E1A/E1B genes. If stable packaging cell lines were made from 293 cells, the use of E1A/E1B defective adenovirus should suffice in providing helper functions for AAV vector production. E1A/E1B-defective adenovirus has been widely used as a gene therapy vector in humans because its safety profile is much better than that of wild type adenovirus. For this reason, extensive efforts have been made to generate 293 cell-based AAV packaging cell lines. However, no such cell line has been generated to date that offers both stability and high-productivity (Chadeuf et al. 2000; and Yang, Q., Chen, F., and Trempe, J. P. 1994. Characterization of cell lines that inducibly express the adeno-associated virus Rep proteins. J Virol 68:4847-56).

Clark et al. have attempted to generate AAV packaging cell lines in parallel from both HeLa cells and 293 cells (Clark et al. 1995). To their credit, they have succeeded with HeLa cells in generating stable and high-titer AAV packaging cell lines. However, they were unable to generate such cell lines with 293 cells. Similar effort were also made by Chadeuf et al. in an attempt to generate AAV packaging cell line from both HeLa cells and 293 cells (Chadeuf et al 2000). Again, they succeeded with the HeLa cells, and also partially succeeded in obtaining a 293 cell-based AAV packaging cell line named 293RC21. However, this cell line gave rise to very low titers of AAV vectors (4×10¹⁰ v.g. particles from 20×15-cm plates cells). Furthermore, the capability for AAV vector production decreased by more than 10 fold after the cell line was passaged in culture for one month. Subcultivates of 293RC21 were unstable with low vector yield, in sharp contrast to their corresponding HeLa cell-based packaging cell line HeRC32, which remained stable and produced high-yields of virus (Id.).

Previous failures to generate 293 cell-based AAV packaging cell lines were primarily due to the constitutive expression of the Ad E1A gene in 293 cells that activates the AAV Rep gene promoters p5 and p19 (Shi, Y., Seto, E., Chang, L. S. and Shenk, T. 1991. Transcriptional repression by YY1, a human GLI-Kruppel-related protein, and relief of repression by adenovirus E1A protein. Cell 67:377-88), which in turn produced four Rep proteins. The latter have been proven to be cytostatic (Yang et al. 1994) and cytotoxic (Schmidt, M., Afione, S. and Kotin, R. M. 2000. Adeno-associated virus type 2 Rep78 induces apoptosis through caspase activation independently of p53. J Virol 74:9441-50), preventing the formation of stable cell lines. On the other hand, both AAV p5 and p19 promoters in HeLa cell-based packaging cell lines are silenced, similar to the latent AAV infection, where no AAV gene expression could be detected. While silencing of the AAV promoters made it possible for HeLa cell-based packaging cell lines to grow, such promoter silencing could not be achieved in 293 cells because of the transcription activator E1A acting on AAV promoters.

Several attempts to overcome Rep-mediated cytostatic and cytotoxic effects in 293 cells have been focused on the control of gene expression of the two larger Rep proteins, Rep78 and Rep68, because manipulation of p5 promoter was relatively convenient. Yang et al. made a 293 cell line with inducible Rep78 and Rep68 gene expression by replacing the p5 promoter with an inducible promoter (Yang et al. 1994). However, in that cell line the promoter p19 products Rep52 and Rep40, which are essential for virus packaging, were barely detectable even under induced conditions or after adenovirus infection. There also was no AAV capsid gene in that cell line.

In another effort to control Rep78 and Rep68 expression in 293 cells, Ozawa and colleagues (Ogasawara, Y., Mizukami, H., Urabe, M., Kume, A., Kanegae, Y., Saito, I., Monahan, J. and Ozawa, K. 1999. Highly regulated expression of adeno-associated virus large Rep proteins in stable 293 cell lines using the Cre/loxP switching system. J Gen Virol 80(9):2477-80) replaced the p5 promoter with a strong exogenous promoter followed by a “stuffer” sequence containing a blasticidin S resistance gene inserted before the Rep coding sequence. The removal of the stuffer sequence by Cre/loxP-mediated recombination, by Ad-Cre infection, turned on Rep78 and Rep68 gene expression. However, the Rep52, Rep40 and Cap genes could not be induced in the cell line even after Ad-Cre infection, or after a different Ad infection. The authors attributed the failed gene expression of those essential AAV proteins to their deleterious effects, which prevented the growth of 293 cells that could have a complete set of functional Rep and Cap proteins (Ogasawara et al. 1999). An antisense DNA strategy designed by the same group also was unable to generate a stable and high-titer 293 based AAV packaging cell line, and it was concluded that long-term leaky expression of the Rep gene products may confer a growth disadvantage on cells (Okada, T., Mizukami, H., Urabe, M., Nomoto, T., Matsushita, T., Hanazono, Y., Kume, A., Tobita, K. and Ozawa, K. 2001. Development and Characterization of an Antisense-Mediated Prepackaging Cell Line for Adeno-Associated Virus Vector Production. Biochem Biophys Res Commun 288:62-8).

The major obstacle to generating useful 293 cell-based AAV packaging cell lines is E1A-mediated activation of AAV promoters p5 and p19. These two promoters control the gene expression of four AAV replication proteins (Rep78, Rep 68, Rep 52 and Rep 40), which are known to be cytostatic and/or cytotoxic when expressed at high levels. As a result, both promoters p5 and p19 need to be regulated tightly during packaging cell line growth and highly induced during rAAV vector production. A further obstacle to the tight regulation of p19 is a consequence of the location of this promoter, which is situated within the protein coding region of promoter p5 products, Rep78 and Rep68. Manipulation of the p19 promoter will inevitably cause mutations in the Rep78 and Rep68 coding sequence and may disrupt the structure and functions of these essential Rep proteins.

### SUMMARY

A method is provided for efficient, silent control of gene expression. In the method, an intron containing a disruptable transcription termination sequence (a “terminating intron”) is inserted into a target gene to limit or prevent full-length transcription of the gene. Before the transcription termination sequence is disrupted, transcription of the target gene is terminated at the transcription termination sequence within the intron. To disrupt the disruptable transcription termination sequence, a trans-acting factor for disrupting the transcription termination sequence is introduced into a cell containing the target gene, thereby rendering the transcription termination sequence inactive, permitting full-length transcription of the target gene. The trans-acting factor may be introduced into a cell containing the target gene controlled by the terminating intron by any method, including by gene transfer or by protein transfer.

In one example, the transcription termination sequence is flanked by a pair of recombination sites. In that case, the trans-acting factor is a recombinase that excises or inverts sequences flanked by the recombination sites. Specific examples of recombinase enzymes and recombination sites are Cre/LoxP and FLP/FRT. This embodiment also is referred to as a dual splicing switch because two splicing events occur when the target gene is expressed.

The terminating intron is useful in controlling expression of virtually any gene. However, one use is to control expression of cytotoxic or cytostatic genes so that those genes can be propagated in a cell line. Examples of cytostatic or cytotoxic genes are the AAV Rep gene (Rep) and autonomous parvovirus non-structural (NS) genes. By positioning a terminating intron in the reading frame shared by all four Rep proteins, expression of all four Rep genes can be controlled effectively. When the transcription termination switch is active, levels of Rep production are low enough to permit propagation of cells bearing the inactivated Rep gene. When the transcription termination sequence is disrupted, the Rep genes are fully and correctly expressed.

A nucleic acid is therefore provided containing a terminating intron having a disruptable transcription termination sequence. The terminating intron may be inserted into an expressed sequence of a target gene to control expression of the target gene. The target gene may be a Rep gene or an NS gene. In one example, the disruptable transcription termination sequence is excisable, typically by a recombinase, such as Cre or FLP.

The transcription termination sequence can be any sequence that can terminate transcription to a desired level. A typical transcription termination sequence is a polyA (polyA) sequence. Another transcription termination sequence is a gene. Combinations of a gene and one or more polyA sequences also display synergy. An example of such a combination is three polyA sequences followed by a gene (the gene is 3′ to, or downstream of the polyA sequences). For convenience of establishing a cell line with the target gene under control of the dual splicing switch, the gene of the terminating intron contains a selectable marker, such as, without limitation, hygromycin- or puromycin-resistance genes.

Also provided is a cell or cell line including a nucleic acid containing the above-described terminating intron, typically positioned in an expressed sequence of a target gene. Similarly, a non-human transgenic animal is provided including a target gene under control of a terminating intron.

A method for expressing a gene in a cell is provided that includes the step of contacting a nucleic acid in the above-described cell or cell lines with a trans-acting agent for disrupting the transcription termination sequence.

A method for producing a rAAV transducing unit, or particle, also is provided. The method includes the step of introducing a trans-acting factor, such as a recombinase enzyme, into a cell containing a first nucleic acid sequence including AAV sequences encoding a Rep gene and any additional AAV proteins necessary in trans for production of an rAAV transducing unit, such as the Cap gene. The Rep gene contains a terminating intron positioned in a coding sequence shared by Rep78, Rep68, Rep52 and Rep40 proteins. In one embodiment, the intron includes a transcription termination sequence flanked by a pair of recombination sites. The cell also may contain a template for a packageable rAAV genome. Similarly, a method for producing recombinant parvovirus particles is provided that includes the step of introducing a trans-acting factor, such as a recombinase enzyme, into a cell containing a first nucleic acid sequence including sequences for an NS gene encoding two or more proteins from a shared reading frame and any additional parvovirus proteins necessary in trans for production of a recombinant parvovirus transducing unit. The non-structural gene contains the above-described terminating intron positioned in the shared reading frame of the NS gene. The cell also may contain a template for a packageable recombinant parvovirus genome.

### BRIEF DESCRIPTION OF THE DRAWINGS

FIGS. 1A-C show construction and function of two dual spicing switch plasmids for gene expression control of Lac-Z and AAV Rep genes. FIG. 1A shows construction of dual spicing Lac-Z plasmids. pLacZ-Int: Lac-Z gene containing an hCG intron insertion in the coding sequence (Sun, L., Li, J. and Xiao, X. 2000. Overcoming adeno-associated virus vector size limitation through viral DNA heterodimerization. Nat Med 6:599-602). The Bgl II site is for cloning purpose. pLacZ-Int-3A: a triple SV40 polyA cassette (3A) flanked by two LoxP sequences was inserted into the Bgl II site in the hCG intron of pLacZ-Int to block Lac-Z gene transcription. pLacZ-Int-Hyg: The Hyg^{r} gene flanked by two LoxP sequences was inserted into the BglII site of pLacZ-Int. pLacZ-Int-3A-Hyg: The triple SV40 polyA together with Hyg^{r} gene flanked by two LoxP sequences was inserted into the Bgl II site of the pLacZ-Int. FIG. 1B shows construction of dual splicing switch AAV packaging plasmids. pXX2 is an AAV type-2 packaging plasmid (Xiao el al. 1998). pXX-Int-3A-Hyg: the 3.2 kb termination cassette (Int-3A-Hyg) from plasmid pLacZ-Int-3A-Hyg was inserted into the shared Rep coding sequence downstream of promoter p19 to block Rep gene transcriptions. pXX-Int-3A-Puro: the Hyg^{r} gene was replaced by puro^{r} gene for additional selection. pXX-Int-3A-Hyg-GFP: an AAV GFP vector which contains a CMV promoter-driven EGFP gene and a Neomycin resistant gene was inserted into the Sse I site of pXX-Int-3A-Hyg plasmid. FIG. 1C shows activation of Lac-Z gene controlled by the dual splicing switch.

FIGS. 2A are photomicrographs showing Lac-Z gene expression from dual splicing controlled Lac-Z constructs. Different plasmids were transfected into the 293 cells, as indicated. FIG. 2B is a graph showing β-galactosidase enzyme activity in 293 cells from dual splicing controlled LacZ constructs. Data represent three parallel experiments. For plasmid pLacZ-Int-3A-Hyg, the induction experiment was achieved by infection with Ad-Cre (m.o.i.=5), and the control group was infected with Ad-GFP (m.o.i.=5).

FIG. 3 shows autoradiographs of Western analysis of Rep gene induction by Ad-Cre infection. Rep gene induction by Ad-Cre infection either in plasmid transfection experiments (lanes 1 to 4) or in cell lines (lanes 5 to 8). Plasmids pXX-Int-A3-Hyg-2 (lanes 1 & 2) and plasmid pXX-Int-A3-Hyg-8 (lanes 3 & 4) had no Rep gene expression in transfection (lanes 1 & 3) but showed high-level Rep gene expression at 48 hours after Ad-Cre infection (lanes 2 and 4). Lane 5 to Lane 8 are examples of Cre-inducible 293 cell-based AAV packaging cell lines. After Ad-Cre infection for 48 hours, two clones showed very high Rep gene expression (lanes 5 and 7) and two clones showed modest Rep gene expression (lanes 6 and 8).

FIG. 4 is an autoradiograph of a Northern blot showing the time course of Rep and Cap gene expression in 293 based AAV-GFP packaging cell line (293-GFP-145) following Ad-Cre infection. Stable AAV packaging cell line 293-GFP-145 was infected with Ad-Cre at 5 m.o.i. Cell pellets were harvested at different time intervals and analyzed by western blot using anti-Cap antibody (top panel) or anti-Rep antibody (bottom panel). A control virus Ad-GFP was also used to infect the cells (lane 7). A transfection experiments was done as another control with packaging plasmid pXX2 and mini-Ad plasmid pXX6 (lane 8) (Xiao et al. 1998).

FIG. 5 is a graph showing that the multiplicity of infection by Ad-Cre showed minimal effects on the yields of AAV-GFP vector from cell line 293-GFP-145.

FIG. 6 is an autoradiograph showing Southern analysis for replication competent AAV (rcAAV) generated during production of AAV-GFP vectors. Up to 10⁹ viral genome particles of highly purified AAV-GFP, generated either from the 293-GFP-145 cells or from triple plasmid transfection method, were used to infect 293 cells, which were co-infected with wild-type adenovirus at 5 m.o.i. to provide helper functions for rcAAV amplification. As a positive control, wild-type AAV also was used similarly to infect the 293 cells at multiplicities ranging from 1 to 1000 infectious units along with wild-type adenovirus co-infection. Low-molecular-weight DNA samples from the second round of amplification were isolated and subjected to Southern analysis with an AAV Cap gene fragment as the probe.

FIG. 7 is an electron micrograph of AAV viral particles produced in 293-GFP-145 cell line and purified by heparin sulfate column purification. The original magnification was 50,000×. The particles were diluted to 2×10¹² vector genomes per ml for detection.

FIGS. 8A-C depict PCR analysis of AAV Rep gene structure before and after Ad-Cre and LoxP mediated DNA splicing. FIG. 8A provides a schematic illustration of the Rep gene structure and expected sizes of PCR products. Primer PCR-F and primer PCR-R-Rep were located in Rep gene respectively before and after the inserted intron. Primer PCR-R-Int was located in the 3′ end of the intron. FIG. 8B is an autoradiograph showing gel electrophoresis of PCR products with primers PCR-F and PCR-R-Int. Total cellular DNA was isolated from cell line 293-GFP-145 (lanes 1, 2 & 3) or control 293 cells (lane 4) with or without indicated adenovirus infection. The DNA was subjected to PCR amplification and gel separation. FIG. 8C is an autoradiograph showing gel electrophoresis of PCR products with primers PCR-F and PCR-R-Rep. Total cellular DNA (lanes 5, 7, 8 and 9) or episomal DNA (lane 6) was isolated from different cells with or without indicated adenovirus infection. Cell line XX-GFP-53 (lane 7) contain wild-type Rep sequence and yielded a 520 bp PCR product, while cell line 293-GFP-145 contain an inserted intron sequence and a LoxP site (after Cre mediated splicing) and yielded a 910 bp PCR product from both total cellular DNA (lane 5) and episomal DNA (lane 6).

FIG. 9 provides autoradiographs depicting Southern analysis of AAV Rep-Cap gene amplification in AAV packaging cell lines. Total cellular DNA (15 μg) was isolated from different cell lines 48 h after indicated adenovirus infection. Episomal DNA (lane 6) was isolated from 293-GFP-145 cells after Ad-Cre infection. All DNA samples were digested with Pst I to drop a 2.3 kb internal fragment of the AAV Cap gene. The same DNA fragment purified from AAV plasmid was used as a probe for Southern hybridized. The DNA copy number standards were set at 5, 50, and 500 Rep-Cap genome per cell by spiking 0.13, 1.3, and 13 ng of plasmid pXX-Int-3A-Hyg into 15 μg of plain 293 cell DNA. The detected band corresponds to the expected 2.3 kb Cap gene fragment.

FIGS. 10A-D provide non-limiting examples of nucleotide sequences for LoxP (FIG. 10A, SEQ ID NO: 1), encoding CRE (FIG. 10B, GenBank Accession No. X03453, SEQ ID NO: 2), for FRT (FIG. 10C, SEQ ID NO: 3) and encoding FLP (FIG. 10D, SEQ ID NO: 4).

### DETAILED DESCRIPTION

Described herein is a gene expression control method and embodiments thereof. In the method, a transcription termination sequence is inserted into an intron in an expressed sequence of a target gene to disrupt transcription of the target gene, thereby forming a terminating intron. The transcription termination sequence is disruptable by a trans-acting agent to be introduced by any method into a cell in which the target gene is harbored. For example and without limitation, the intron may contain multiple poly A sequences and, optionally, a second gene, flanked by recombination sequences, for instance LoxP recombination sequences. When a suitable recombinase, such as Cre, is added to the cell containing the target gene, the polyA sequences and the second gene are excised (or inverted, depending on the relative orientation of the recombination sequences).

In use, the messenger RNA of the target gene is prematurely terminated by the presence of the transcription termination sequence of the terminating intron, thereby blocking the gene expression. By disruption of the transcription termination sequence, transcription will continue and full-length mRNA will be generated from the target gene. After RNA splicing, the remainder of the inserted intron is precisely removed from the fill-length mRNA, restoring the protein coding sequence of the target gene.

As used herein, a “gene” is an operative genetic determinant in its broadest sense. A gene includes an “expressed sequence” that encodes a protein or is transcribed into a functional RNA product. A typical gene includes an expressed sequence, along with operably linked regulatory sequences, including, but not limited to, promoters, enhancers, operators and terminators. Two sequences are “operably linked” if they are arranged in cis to act in an expected manner in relationship to each other. In a gene, regulatory sequences are operably linked in a manner sufficient to cause correct and/or desired transcription of the expressed sequence in a cell. Promoters can be, for example and without limitation, constitutive or semi-constitutive (for example, CMV and RSV promoters) or tissue-specific promoters (for example, a muscle creatinine kinase (MCK) promoter). The terms “expression” or “gene expression,” and like words and phrases, mean the overall process by which the information encoded in a nucleic acid, typically a gene, is converted into a ribonucleic acid and/or a protein, or a post-translationally modified version thereof, and/or an observable phenotype.

The expressed sequence of a gene may be obtained, synthesized and/or isolated from, for instance and without limitation, a genomic DNA library, cDNA library, vector, plasmid, cosmid, phage or any other gene source known in the art by any method, including direct chemical synthesis and PCR synthesis. The expressed sequence also may be of any species. A gene may code for any expression product and, where the expression product is a protein, the gene may contain two or more shared reading frames for expression of two or more proteins. As shown in the Examples below, the target gene can be a gene that is toxic to a cell, to be activated by disrupting the transcription termination sequence when desired, for instance when needed for preparing rAAV particles.

For purposes herein, the various serotypes of AAV and variations and derivatives thereof, such as, without limitation, recombinant versions of any AAV serotype or serotypes are considered to be “AAV.” Derivatives, analogs or homologues of the AAV genome, or any portion thereof, including ITR, Rep and Cap sequences also are considered to be AAV sequences.

As used herein, an “intron” is broadly defined as a sequence of nucleotides, typically in an expressed sequence of a gene, that is removable by RNA splicing. “RNA splicing” means the excision of an intron from a pre-mRNA to form a mature mRNA. Insertion of DNA containing (encoding) an intron into an expressed sequence can be accomplished by any method known in the art.

A typical intron contains a 5′ splice site or junction, a splice acceptor or branch point, and a 3′ splice site or splice junction. The term “5′ splice site” or “5′ splice junction” means the exon-intron junction between the 3′ end of a 5′ fragment of a gene and the 5′ end of the intron, and includes the sequences at the 5′ end of the intron that are required for RNA splicing. The term “splice acceptor” or “branch point” refers to the nucleotide, usually adenosine, located approximately 20-50 bp from the 3′ splice site that helps form the lariat structure during the first trans-esterification reaction during RNA splicing. The term “3′ splice site” or “3′ splice junction” means the exon-intron junction between the 5′ end of a 3′ fragment of a gene and the 3′ end of the intron, and includes the sequences at the 3′ end of the intron that are required for RNA splicing. A typical intron includes consensus sequences that are typical nucleotides in, or adjacent to either the 5′ or 3′ splice junction or the splice acceptor that are required for RNA splicing; these sequences usually are either invariant or highly conserved as shown, without limitation, in Lodish et al., Molecular Cell Biology, 4th ed., W. H. Freeman & Co. 2000, p. 416, FIGS. 11-14, which is incorporated herein by reference.

In reference to any nucleotide or protein sequence described herein, including without limitation: “derivatives” of genes and genetic elements—such as without limitation: promoters; enhancers; operators; terminators; recombination sequences; AAV ITR sequences; introns; expressed sequences; gene expression products terminating introns and dual splicing switches—are nucleotide or protein sequences that include sequence insertions, deletions or substitutions that do not substantially alter the function of the nucleotide or protein sequence. Derivatives may have equal, lower or higher functionality, so long as the overall function and specificity of the sequence is substantially retained. For example, for any AAV ITR sequence, nucleotides may be inserted, deleted or substituted to produce a derivative ITR, so long as the derivative ITR retains the ability to facilitate AAV or rAAV genome replication, packaging and host integration capabilities. “Analogs” are nucleic acids or proteins that contain one or more a typical (are not deoxyribo- or ribo-nucleosides and/or do not contain adenine, guanine, cytosine, thymine/uracil bases) nucleoside or amino acid residues. “Homologues” are equivalent proteins or nucleic acids found both in different members of the same species (including alleles) and in members of different species. As with derivatives, analogs and homologues do not substantially alter the function of a given protein or nucleic acid and may have equal, lower or higher functionality as compared to a given protein or nucleic acid.

As used herein, a “termination intron” is an intron containing a disruptable transcription termination sequence inserted into a target gene. The disruptable transcription termination sequence contains a sequence or sequences that facilitate trans-activated disruption of the transcription termination function of the transcription termination sequence. In one embodiment, the transcription termination sequence is disruptable by recombination, in that it is excisable or invertable because it is flanked by recombination sites. An intron containing this excisable transcription termination sequence has the following structure:

Intron 5′ splice junction-REC-TTS-REC-splice acceptor-3′ splice junction

Where REC is a recombination site such as LoxP, TTS is a transcription termination sequence as described herein, and the 5′ splice junction, splice acceptor and 3′ splice junction are at least minimal intron splicing sequences, as defined above. When the termination sequence is disruptable by DNA splicing event(s), such as by recombination, the terminating intron is referred to as a “dual splicing switch” because two splicing events occur (DNA and RNA splicing) before the transcribed sequence is useful either as functional RNA or in translation.

A “transcription termination sequence” is any nucleic acid sequence that would cause a desired level of premature termination of transcription of a target gene when inserted into the coding sequence of the target gene as a terminating intron as defined herein. By “premature termination,” it is meant termination of transcription of a target gene before the expressed sequence of the target gene is transcribed in a manner that would lead to the production of a functional expression product of the target gene. A transcription termination sequence may include, without limitation, one or more polyA sequences and a second gene, in various combinations or orientations. Although very tight transcriptional control may be maintained using the combination of three polyA sequences followed by a second gene, as shown in the examples below, a desirable level of transcription termination may be effectively achieved by a variety of combinations of elements, with polyA elements located before the second gene, after the second gene, or both. The second gene may be omitted, in which case the transcription termination sequence typically, but not necessarily, includes two or more polyA sequences. However, as discussed above, the number of polyA sequences, the presence or absence of a second gene, and the respective locations of these termination elements determine the strength of the premature termination for any given combination of target gene and dual splicing switch.

The required strength of premature termination depends on the system in which the dual splicing switch is used. In most cases, strong premature termination is desirable. However, certain gene expression systems either may tolerate or may require weaker termination, and, therefore, the weaker transcription termination sequences might be preferred in some instances. Stronger termination sequences would be necessary where expression of the target gene is harmful to the cells into which the target gene is transferred, or where low levels of expression interferes with analytical method(s) or cell selection method(s). As shown herein, for use in a packaging cell line for rAAV particles, or for other parvoviruses, when the dual splicing switch is inserted into the Rep gene, or the NS gene, strong premature termination is required due to the cellular toxicity of the Rep and NS proteins.

The transcription termination sequence that is part of the terminating intron described herein is considered to be “disruptable,” meaning that the transcription termination activity of the transcription termination sequence may be inactivated by a trans-acting agent. The transcription termination sequence of a dual splicing switch is “excisable” when it is flanked by recombination sequences, or by other sequences that facilitate excision and re-splicing of DNA containing the transcription termination sequence by a recombinase, or another factor, provided in trans. Any nucleic acid sequence or sequences that facilitate disruption of the transcription termination sequences by action of, or as a result of the presence of a factor provided in trans is/are suitable and, when combined with the transcription termination sequences form a “disruptable transcription termination sequence.”

A transcription termination sequence is considered to be disrupted when it is “inactivated,” which means that transcription of the target gene is restored to a desired degree, typically, but not necessarily, to a point where levels of full length transcription of the target gene approach, reach or surpass levels achievable if there were no transcription termination sequences in the intron. Disruption can be achieved a number of ways including by insertion, inversion, modification, substitution or deletion of certain sequences. By “excised,” “excisable” and like terms, it is meant that the DNA transcription termination sequences are removed or removable, for instance by recombination as described herein, so as to inactivate the transcription termination sequences to a desired degree. Nucleic acid sequences for facilitating disruption of the transcription termination sequence act, along with an appropriate trans-acting factor, without disrupting either: 1) function of the intron splicing mechanism of the intron in which the disruptable transcription termination sequence is/was contained or 2) correct and/or desired expression qualities of the target gene.

A “selectable marker” is a gene or nucleic acid sequence that permits selection of a cell containing (or not containing) that marker. Examples of selectable markers are well known in the art. Non limiting examples of selectable markers are antibiotic resistance genes including, without limitation, hygromycin-resistance (hyg^{r}), puromycin-resistance (puro^{r}) and blasticidin S-resistance (bsr^{r}) genes.

A “recombination sequence” is a nucleotide sequence that permits disruption of the transcription termination sequence by a recombination event when a recombinase is provided in trans. Non-limiting examples of recombination sites are LoxP and FRT. The recombination sites are inserted into the dual splicing switch flanking a transcription termination sequence, or a portion thereof, to be excised by a recombination event. The target gene is activated by the addition of a recombinase (Cre or FLP in the case of LoxP and FRT, respectively). Optionally, the recombination sites may be oriented to invert the sequences located therebetween when a recombinase is added. Orienting the recombination sequences to invert the intervening sequences only would function with termination sequences that are not bi-directionally functional (such as some polyA sequences). Inversion is reversible because the recombination sequences remain in close proximity, in cis, and would revert so long as a recombinase enzyme is present, leaving a mixed population of inverted and non-inverted sequences. Notably, reversion is less common when the recombination event results in excision of the termination sequences because the recombination sites are no longer located on the same DNA molecule—that is, a much rarer trans event would need to occur before reversion.

A number of methods exist for introducing trans-acting factors, such as recombinase enzymes into a cell containing the target gene. In a first method, a nucleic acid containing a gene encoding a recombinase is transferred into the cell. Any gene transfer method is applicable, such as, without limitation, viral-mediated (for example, Adenovirus-mediated transduction) or non-viral-mediated (liposome, Calcium phosphate-mediated, electroporation, etc.) gene transfer methods. As shown below, in its use in a packaging cell line for rAAV particles, the Cre enzyme may be added in trans by transduction with an Ad-Cre-recombinant virus.

In another embodiment, the trans-acting factor is modified for direct protein transfer into cells containing a gene controlled by a dual splicing switch. The essential principle for protein delivery is the fusion of a small peptide sequence to a large protein. This small peptide is any peptide that facilitates protein transfer into a cell. One example of such a small peptide is a peptide from HIV virus tat protein (Schwarze, S. R., Ho, A., Vocero-Akbani, A. and Dowdy, S. F. In vivo protein transduction: delivery of a biologically active protein into the mouse. Science. Sep. 3, 1999;285(5433):1569-72, amino acid sequence: YGRKKRRQRRR (SEQ ID NO: 5)), which has been shown to facilitate delivery of Lac-Z protein into an entire animal, including brain tissues, through blood vessels. Investigators also have fused this same peptide with Cre and have delivered Cre protein into cells and tissues (Peitz, M., Pfannkuche, K., Rajewsky, K. and Edenhofer, F. Ability of the hydrophobic FGF and basic TAT peptides to promote cellular uptake of recombinant Cre recombinase: a tool for efficient genetic engineering of mammalian genomes. Proc Natl Acad Sci USA. Apr. 2, 2002;99(7):4489-94.).

Generally, as an extension of direct protein transfer technology, a peptide fragment that is highly positively charged (for example, contains sufficient amounts of lysine and/or arginine) can cross a cell membrane directly, and, when fused to a protein, has the potential to drag the fusion protein into a cell (Wender, P. A., Mitchell, D. J., Pattabiraman, K., Pelkey, E. T., Steinman, L. and Rothbard, J. B. The design, synthesis, and evaluation of molecules that enable or enhance cellular uptake: peptoid molecular transporters. Proc Natl Acad Sci USA. Nov 21, 2000;97(24):13003-8). Even unmodified Cre protein can be directed delivered into the host cells to achieve recombination activities (Will, E., Klump, H., Heffner, N., Schwieger, M., Schiedlmeier, B., Ostertag, W., Baum, C. and Stocking, C. Unmodified Cre recombinase crosses the membrane. Nucleic Acids Res. Jun. 15, 2002;30(12):e59).

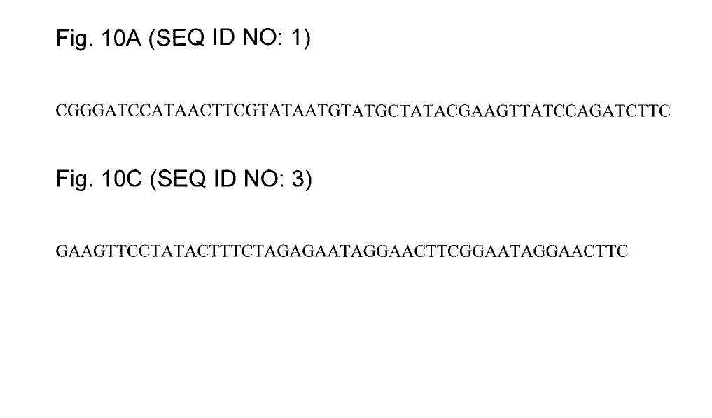

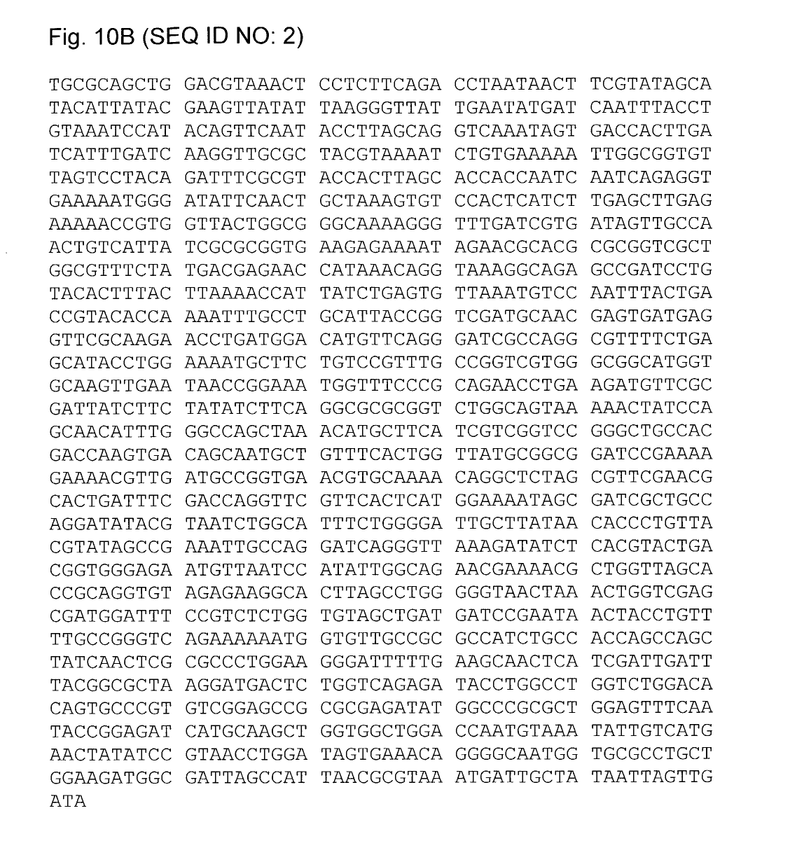

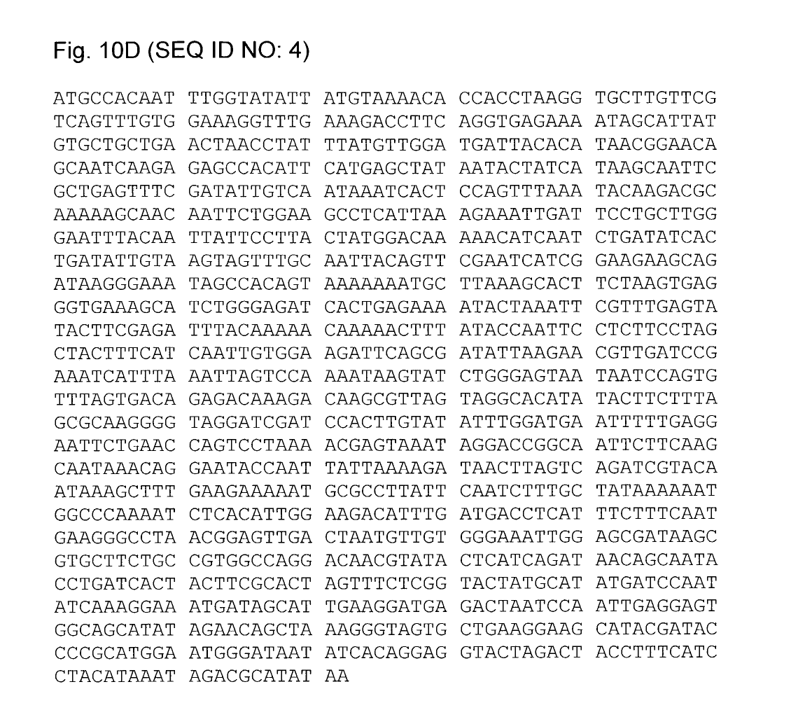

In one embodiment, the terminating intron is a dual splicing switch containing an excisable transcription termination sequence. The excisable transcription termination sequence typically is a sequence containing a transcription termination sequence flanked by recombination sites. The most commonly used and well-characterized recombinase/recombination site combination is the Cre/LoxP combination, however, other recombinase/recombination site combinations may be substituted therefor, including, without limitation, the FLP/FRT combination. The LoxP sequences and the sequences encoding Cre are broadly known (see, for example and without limitation the example of a LoxP sequence provided in FIG. 10A (SEQ ID NO: 1) and GenBank Accession No. X03453, “Bacteriophage P1 cre gene for recombinase protein,” provided in FIG. 10B, (SEQ ID NO: 2)). Similarly, FRT recombination sequences and FLP recombinase coding sequences are known (for example, and without limitation, the FRT sequence provided in FIG. 10C (SEQ ID NO: 3) and the FLP sequence provided in FIG. 10D (SEQ ID NO: 4)).

The target gene containing the terminating intron may be constructed or obtained in any manner. As described below, as with any cloning process, various combinations of restriction digestions, ligations, selective PCR amplifications and other nucleic acid manipulations, as are well know to those of skill in the field of the present invention, may be employed to prepare a desired nucleic acid construct. For example, and without limitation, recombination sites may be added to a plasmid containing an insert containing three polyA sequences to flank the polyA sequences. A gene, such as a selectable marker or an indicator gene (for example, hyg^{r} or puro^{r}), is inserted between a recombination site and the polyA sequences to produce an excisable transcription termination sequence. This excisable transcription termination sequence then may be inserted into an intron in a target gene by standard cloning methods. As shown below, once an intron containing an excisable transcription termination sequence is constructed, the intron may be amplified by PCR using primers containing a restriction site found in the coding region of any given target gene. By this method, the amplified intron can be inserted into the restriction site in the target gene, subjecting the target gene to control by the dual splicing switch.

In one embodiment, the terminating intron is employed in a packaging cell line for production of rAAV transducing units. In this embodiment, a cell line containing an AAV Rep gene under control of a terminating intron is propagated. The terminating intron is positioned in the Rep gene in a reading frame shared by all four Rep proteins, Rep 40, Rep 52, Rep 68 and Rep 78. This insertion point typically falls between the p19 promoter and the p40 promoter. Due to the robust and ubiquitous nature of RNA splicing and termination and that transcription termination is well-studied, there is no explicit requirement to position the terminating intron at any given nucleotide position in the Rep gene, and most choices are expected to be fully functional. In any case, expression of the Rep gene is readily tested, in the manner shown herein, or otherwise.

A packaging cell line for AAV would produce any AAV proteins necessary for the production of rAAV transducing units. These proteins include Rep proteins and Cap proteins. Although the Rep and Cap proteins may be transferred into the packaging cell line separately, they typically are transferred together as a single DNA construct. The construct (typically a plasmid construct) usually contains a substantially complete AAV genome with no inverted terminal repeat (ITR) sequences functional in packaging the AAV genome and with a terminating intron inserted between the p19 and p40 promoters. Optionally, a second terminating intron is inserted into the Cap gene so that little or no Cap protein is produced prior to disruption of the transcription termination sequences. One source, among many, of a substantially complete AAV genome, less ITR sequences, is the plasmid pXX-UF1, described below and elsewhere.

The packaging cell line also typically contains a nucleic acid template for a packageable rAAV genome. The template includes AAV ITR sequences which flank a filler sequence that typically is a gene, or a portion thereof. The rAAV genome is packageable in that it can be packaged into a rAAV transducing unit. The ITR sequences typically range in length from 140-170 nucleotides each, with the AAV2 ITR being 145 nucleotides. The ITR may be from, or derived from, any AAV ITR and is useful so long as it facilitates replication, packaging and integration of the rAAV genome.

In a related embodiment, the viral system is a member of the Family Parvoviridae (“parvovirus”), typically of the SubFamily Parvovirinae and most typically a member of the Genera Parvovirus, Erythrovirus and Dependovirus. In the case of autonomous parvoviruses, for example, the cytotoxic non-structural gene can be interrupted by the dual splicing switch in a similar manner as the AAV Rep gene, permitting construction of a cell line containing the non-structural gene. Parvovirus species that may be of use include, without limitation, minute mice virus, canine parvovirus, bovine parvovirus, porcine parvovirus, Hi, LuIII and B19 parvoviruses.

The AAV (or parvovirus) packaging cell line may be prepared by any transformation and selection method. Though 293 cells are one cell line that is particularly suited to rAAV production, other cell lines, especially easily transformable cell lines naturally or artificially expressing Ad E1A and E1B genes, would be suitable. The cells are stably transformed with a first nucleic acid containing at least a Rep gene under control of a terminating intron, such as a dual splicing switch. The first nucleic acid typically also would contain AAV Cap genes. In one embodiment, a dual splicing switch contains a selectable marker, such as puro^{r} and hyg^{r}, which would permit selection of cells containing the inactivated Rep gene. Alternately, the first nucleic acid contains a selectable marker not within the Rep gene or the terminating intron, also permitting selection of cells containing the nucleic acid.

At any time before, during or after establishment of a cell line containing the first nucleic acid, the cell to contain or containing the first nucleic acid is stably transformed with a second nucleic acid containing a template for a rAAV genome, as described above. The desired end result being a cell stably transformed with both the first nucleic acid and the second nucleic acid. Logically, but not necessarily, it would be preferable to first establish a cell line stably transformed with the first (Rep-disabled) nucleic acid, facilitating creation of numerous packaging cell lines by transformation with a variety of nucleic acids containing different rAAV genome templates.

Once a stable packaging cell line is produced, the cell can be transduced with an E1A/E1B deficient Adenovirus containing a gene encoding a trans-acting factor for disrupting the transcription termination sequences of the terminating intron, such as Ad-Cre. In cases where the trans-acting factor is delivered by direct protein delivery, as described above, necessary Adenovirus factors may be added by infection with E1A/E1B-deficient Adenovirus, or by transfer of necessary Ad genes by other methods as are well-known in the art.

In a further embodiment, the intron containing the excisable transcription termination sequence is inserted into the non-structural gene sequences of an autonomous parvovirus, such as the B19 NS gene, in a location sufficient to down-regulate expression of the non-structured gene so that a cell line can be transformed with a construct manufactured in this manner and cells transformed in this manner can be propagated as a cell line. An autonomous parvovirus packaging cell line can be produced by the methods described herein, containing a dual splicing switch-inactivated non-structural gene and any additional genes necessary in trans for packaging a recombinant parvoviral genome, including, but not limited to capsid genes. Heretofore, it has not been possible to make cell lines for the production of recombinant autonomous parvoviruses due to the extreme cytotoxicity of their non-structural proteins to the host cell and that these genes are very actively expressed in transformed cell lines. By using the techniques described herein, the toxicity of the non-structural genes can be avoided.

The technology described herein is an effective gene activation method that can find use in a variety of systems, such as in transgenic technology, for instance when it is desirable to induce expression of a specific gene in a temporal and spatial manner. For example, a transgenic organism may be produced having a target gene inactivated in the manner described herein and a gene encoding a recombinase enzyme having a tissue-specific and/or developmentally-specific promoter. At a specific developmental stage and/or in a particular tissue system, a target gene then may be activated. The benefit of this over traditional transgenics is that the activated target gene may include promoters or other regulatory sequences that are not necessarily tissue- and/or developmentally-specific, permitting targeted expression of any gene in any desired manner. For instance, tissue- and/or developmentally-specific expression may be realized using constitutive or semi-constitutive promoters. Alternately, a transgenic organism may be produced having the inactivated target gene and the recombinase enzyme may later be added through gene transfer methods such as, without limitation, viral-mediated gene transfer and transfection or protein transfer methods. In an additional embodiment, as described in further detail above, a recombinase protein itself, such as Cre recombinase or FLP recombinase, also can be directly delivered into the cells, tissues, or organisms to activate the target gene, which is previously inactivated. The methods are not limited to transgenic organisms. An inactivated gene may be introduced into an organism or cells, tissue or organs of an organism, plant or animal, by any other gene transfer method to be later activated by addition of a recombinase enzyme, typically by gene transfer methods or direct protein delivery methods.

### EXAMPLES

The terminating intron system initially was tested on the β-galactosidase (Lac-Z) gene. By comparing X-gal staining and Lac-Z enzyme activities for a variety of constructs, a dual splicing system was developed that could tightly block the gene expression of the β-galactosidase coding sequence. Further, Lac-Z gene expression could be fully restored after the Cre gene was introduced into the cells.

In an effort to simultaneously block gene expression of all four AAV Rep proteins, a dual splicing switch was inserted in AAV Rep gene coding sequences shared by all four Rep proteins. The dual splicing switch tightly controlled expression of all Rep proteins. As a result, 293 cell-based AAV packaging cell lines were readily obtained that were both highly stable and that produced high vector yields. Upon induction of AAV Rep gene expression by an E1A/E1B/E3-deleted adenovirus carrying the Cre gene, the 293 cell-based AAV packaging cell lines surpassed the yields of transfection methods as well as the yields of HeLa cell-based cell lines.

Plasmid Construction

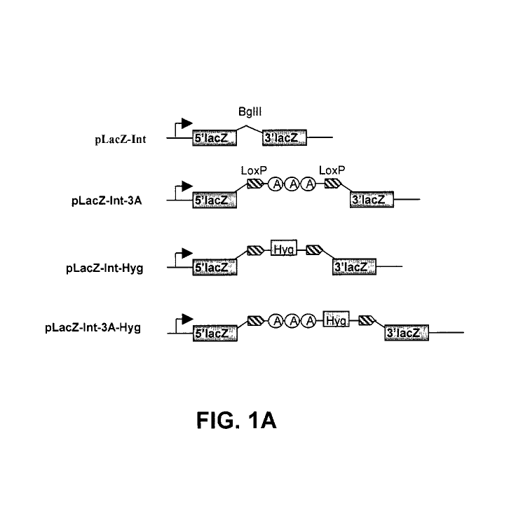

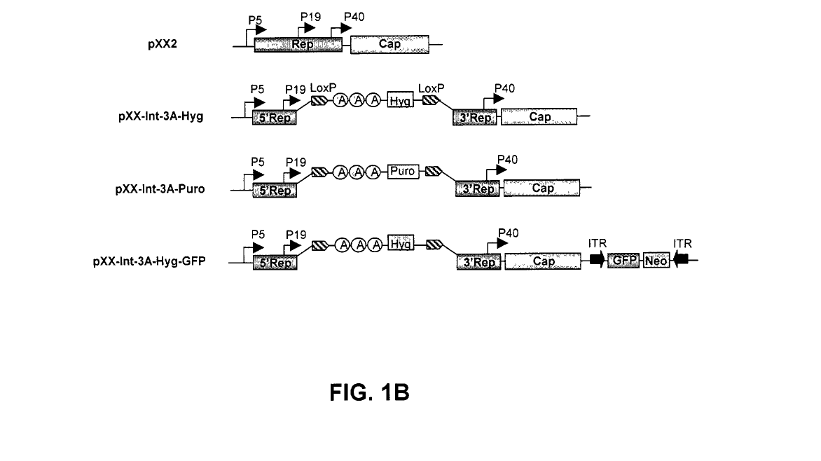

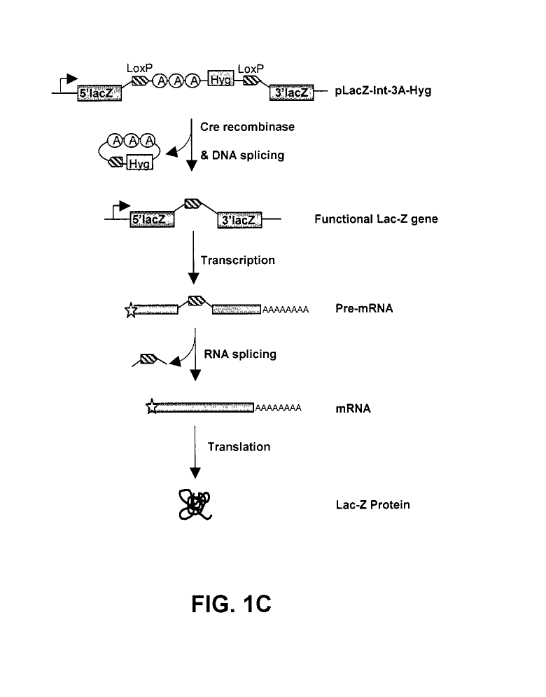

To construct plasmid pLacZ-Int-3A (FIG. 1A), a triple SV40 polyA cassette from plasmid pSVA3 (Maxwell, I. H., Harrison, G. S., Wood, W. M. and Maxwell, F. 1989. A DNA cassette containing a trimerized SV40 polyA signal which efficiently blocks spurious plasmid-initiated transcription. Biotechniques 7:276-80) was excised by BamH I and Bgl II digestion and subcloned into the BamH I site of Bluescript (Stratagene, Calif.) by sticky-end ligation. A LoxP site (CGGGA TCCAT AACTT CGTAT AATGT ATGCT ATACG AAGTT ATCCA GATCTTC) (SEQ ID NO: 1) then was respectively inserted into the Spe I site and the EcoR V site (blunt end ligation) flanking the triple polyA cassette, generating plasmid p2LoxP-SVA3. This plasmid was confirmed by DNA sequencing and revealed direct-repeat orientation of the two LoxP sites. Subsequently, the 2LoxP-SVA3 cassette was excised by Xho I and Not I double digestion, filled in by Klenow enzyme, and inserted via blunt-end ligation into the Bgl II site (Klenow filled in) in the intron of plasmid pLacZ-Int (Sun et al. 2000), generating plasmid pLacZ-Int-3A (FIG. 1A). Using the same strategy, a Hygr gene flanked by the LoxP sites was made and inserted into the Bgl II site of plasmid pLacZ-Int, generating plasmid pLacZ-Int-Hyg (FIG. 1A). Finally, to combine both the 3 polyA and the Hygr gene into one cassette, the Ava I and Hind III digested Hyg^{r} fragment (Klenow filled in) from pTK-Hyg (Clontech, Calif.) was initially cloned into the Sma I site of plasmid p2LoxP-SVA3. Subsequently, the 3A-Hgy cassette flanked by the LoxP sites was digested with Xho I and Not I and inserted into the Bgl II site of pLacZ-Int by blunt-end ligation, generating plasmid pLacZ-Int-3A-Hyg (FIG. 1A).

To make the inducible AAV packaging constructs that have the intron, the triple polyA and Hyg^{r} cassette inserted in the Rep coding sequences, the 3.2 kb Int-3A-Hyg fragment was amplified by PCR from plasmid pLacZ-Int-3A-Hyg with two primers (HCG-Int-forward: 5′ GTAAG AAGATCCGAGGTC 3′ (SEQ ID NO: 6) and HCG-Int-reverse: 5′ CCTTGTCCGGTTACCCTGCAG 3′ (SEQ ID NO: 7)). The PCR amplified intron/polyA/Hyg^{r} cassette was inserted into the Rep coding sequences of pXX2 (Xiao et al. 1998) at two different sites (AAV-2 nucleotide number 1022 or 1340 respectively), generating plasmids pXX-Int-A3-Hyg-2 (FIG. 1B) and pXX-Int-A3-Hyg-8. To construct a similar plasmid which contains a puromycin resistant gene rather than the hygromycin resistant gene, a Puro^{r} cassette was excised by Pvu II and BamH I from plasmid pPUR (Clontech). Plasmid pXX-Int-A3-Hyg-2 was then digested with Mlu I and Rsr II to delete the Hgy^{r} gene cassette. The Puro^{r} cassette was then cloned into Mlu I and Rsr II double-digested pXX-Int-A3-Hyg-2 to replace the Hyg^{r} gene, generating plasmid pXX-Int-A3-Puro (FIG. 1B).

To further combine the AAV vector sequence into the above AAV packaging plasmid pXX-Int-A3-Hyg-2, an AAV GFP vector which contains a CMV promoter-driven EGFP gene and a Neomycin resistant gene was inserted into the Sse I site of pXX-Int-A3-Hyg-2, generating plasmid pXX-Int-3A-Hyg-GFP (FIG. 1B).

Cells and Viruses

Ad-GFP, an adenovirus vector carrying an EGFP gene driven by CMV promoter, and Ad-Cre, an adenovirus vector carrying the Cre recombinase gene of P1 phage (Anton, M. and Graham, F. L. 1995. Site-specific recombination mediated by an adenovirus vector expressing the Cre recombinase protein: a molecular switch for control of gene expression. J Virol 69:4600-6) driven by CMV promoter were made by a conventional method (Jiang, X. C., Qin, S., Qiao, C., Kawano, K., Lin, M., Skold, A., Xiao, X. and Tall, A. R. 2001. Apolipoprotein B secretion and atherosclerosis are decreased in mice with phospholipid-transfer protein deficiency. Nat Med 7:847-52). These adenoviruses were E1A/E1B- and E3-defective first-generation Ad vectors.

293 cell lines were propagated in Dulbecco's modified Eagle's medium (DMEM, Invitrogen) supplemented with heat-inactivated 10% fetal bovine serum (FBS, Invitrogen). Stable transfection was done in 293 cells by the calcium phosphate transfection method as previously described (Xiao et al. 1998). Briefly, right before transfection each well of a 6-well-plate of 293 cells was fed with 1.5 ml of fresh Iscove modified Dulbecco medium (Invitrogen) containing 10% FBS without antibiotics. A total of 3 μg of linearized plasmid DNA was dissolved in 125 μl 0.25M CaCl₂ and then quickly mixed with 125 μl of HEPES-buffered saline and added to the cells. Twelve hours after transfection, the medium was replaced with fresh DMEM containing 10% FBS and antibiotics. Two days later, the cells were trypsinized, diluted, and plated onto 15-cm-diameter dishes to allow for outgrowth of single-cell clones. Concentrations of antibiotics for selection and maintenance of drug-resistant cells were respectively as follows: hygromycin (Clontech), 200 μg/ml and 100 μg/ml; G418 (Invitrogen), 800 μg/ml and 200 μg/ml; puromycin (Sigma), 2 μg/ml and 1 μg/ml.

To generate 293 cell lines that contain the tightly controlled Rep-Cap genes, plasmid pXX-Int-A3-Hyg-2 was transfected into 293 cells and selected by hygromycin. To screen for AAV Rep and Cap gene expression from each of the 293 cell clones, 1×10⁵ cells of each individual clone in a well of a 12-well plate were infected with Ad-Cre virus at a multiplicity of infection (m.o.i) of 5. After infection for 48 to 72 hours, the cells were collected by centrifugation. The cell pellets were subjected to Western blot analysis. The cell clones having a high-level of expression of four Rep proteins were selected and further expanded.

To generate 293 cell-based AAV-GFP vector packaging cell lines, plasmid pXX-int-3A-Hyg-GFP was transfected into a 293 cell line (293-XX-DS-19) that already contained high copies of pXX-Int-A3-Hyg-2 from a previous step (see the above section). Single-cell clones were produced by G418 antibiotic selection. To screen for high-titer AAV-GFP vector packaging cell lines, 1×10⁵ cells of each individual clone in a well of a 12-well plate were infected with Ad-Cre at an m.o.i. of 5. After infection for 48 to 72 hours, both the cells and the media (1 ml) were collected, and subjected to 4 cycles freeze-thaw. The cell debris was removed by centrifugation and the cell lysates were titered on 293 cells for AAV-GFP vector yields from each cell line. Clones with yields of AAV-GFP greater than 10⁷ transducing units (t.u) per 1×10⁵ cells (equivalent to 10⁹ t.u./10-cm plate) were selected for further characterization.

To generate 293 cell-based AAV-mini-dystrophin vector packaging cell lines, plasmid pXX-int-3A-puro was co-transfected with an AAV vector plasmid containing the human dystrophin minigene 3990 under the control of an MCK promoter (Wang et al. 2000), into a 293 cell line (293-XX-DS-19) that already contained high copies of pXX-Int-A3-Hyg-2 from a previous step. Single-cell clones were produced by puromycin selection. To screen for high-titer AAV-mini-dystrophin vector packaging cell lines, 1×10⁵ cells of each individual clone were infected in a well of a 12-well plate with Ad-Cre at an m.o.i. of 5. After infection for 48 to 72 hours, both the cells and the media (1 ml) were collected, and subjected to 4 cycles of freeze-thaw. The cell debris was removed by centrifugation. Supernatant of the cell lysates were subjected to DNA dot blot analysis to determine the viral genome particle (v.g.) titers of AAV-mini-dystrophin vector yields from each cell line. Clones with yields of AAV-mini-dystrophin greater than 10¹⁰ v.g. per 1×10⁵ cells (equivalent to 10¹² v.g./10-cm plate) were selected for further characterization.

AAV Vector Production and Purification

To produce AAV vectors using the 293-based cell lines, the cells were infected with Ad-Cre (m.o.i.=5). Two days following infection, twenty 15-cm plates of the cells were pelleted by centrifugation and re-suspended in suspension buffer (PBS saline with 25 mM HEPES and 150 mM NaCl). After three cycles of freeze/thaw, 0.5% deoxycholic acid and 50 units/ml of Benzonase (Sigma) were added into the cells. The above mixture was incubated for 30 min at 37° C. and centrifuged at 10,000 rpm to remove the debris. The supernatant was further clarified by 0.8 μm filter and directly loaded onto a Hitrap-Heparin column using the AKTA purifier (Amersham Pharmacia Biotech). The AAV viral particles were eluted with 400 mM NaCl in PBS (Zolotukhin, S., Byrne, B. J., Mason, E., Zolotukhin, I., Potter, M., Chesnut, K., Summerford, C., Samulski, R. J. and Muzyczka, N. 1999. Recombinant adeno-associated virus purification using novel methods improves infectious titer and yield. Gene Ther 6:973-85). The viral genome particle titer of the AAV vector was determined by DNA dot blot (Xiao el al. 1998).

Western Analysis of AAV Rep and Cap Proteins

Western blots were carried out by previously published methods with modifications (Li et al. 1997). Briefly, the cell pellet from one well of a 6 well plate was lysed in 200 μl of RIPA buffer (10 mM Tris-Cl, pH 8.2, 1% Triton X-100,1% SDS, 150 mM NaCl). The samples were separated by SDS-polyacrylamide gel electrophoresis (PAGE) (10% polyacrylamide) and transferred to a nitrocellulose membrane. After being subjected to blocking in 10% nonfat dry milk in TBS buffer (50 mM Tris-Cl, pH 7.5, 200 mM NaCl) for 1 h, the membranes were incubated at room temperature for 1 h with primary antibodies in TBS containing 0.5% Tween 20. The primary antibody for Rep was a monoclonal antibody which recognizes all four Rep proteins (Hunter, L. A. and Samulski, R. J. 1992. Colocalization of adeno-associated virus Rep and capsid proteins in the nuclei of infected cells. J Virol 66:317-24) and was used at a 1:75 dilution. The primary antibody for Cap proteins was a guinea pig polyclonal antibody against AAV-2 (Braton Biotech, Inc) and was used at a dilution of 1:400. Following primary antibody incubation and rinses, the membranes were incubated with the secondary antibodies at room temperature for 1 h. The secondary antibody for Rep was a goat anti-mouse polyclonal antibody conjugated to horseradish peroxidase (sigma) at a 1:4000 dilution. The secondary antibody for Cap was a rabbit anti-guinea pig polyclonal antibody conjugated with horseradish peroxidase and was used at a 1:4000 dilution. All the antibodies were diluted with 2% dry milk in TBS buffer. After three washes with TBS, the specific protein bands were visualized by chemiluminescence reagent and exposed to X-ray film.

X-Gal Staining and β-Galactosidase Enzyme Activity Assay

The X-Gal staining and β-galactosidase enzyme activity assays were performed as previously described (Sun el al. 2000; and Xiao, X., Li, J. and Samulski, R. J. 1996. Efficient long-term gene transfer into muscle tissue of immunocompetent mice by adeno-associated virus vector. J Virol 70:8098-108). X-gal staining was performed 36 hours after transfection. For LacZ-Int-3A-Hyg plasmid, the Ad-Cre was added at m.o.i. of 5 during transfection to restore the Lac-Z gene expression.

Infection-Based Viral Amplification Assay for rcAAV Detection

Human 293 cells were seeded at 5×10⁶ cells per 10-cm plate. The next day, the cultures were infected with adenovirus (m.o.i.=5) and infected with various concentrations of AAV vector or wild type AAV. After 72 h, the cells were scraped into the medium and collected by centrifugation. The cell pellets were resuspended in 1 ml of PBS and subjected to three cycles of freeze/thaw in a dry ice-ethanol bath, and centrifuged to pellet debris. The clarified crude lysate was incubated at 56° C. for 1 h to inactivate the adenovirus, and half amount of the crude lysate virus (500 ul) was added to a fresh plate of 293 cells with fresh adenovirus (m.o.i.=5) for a second round of amplification. After an additional 72 h, the cells were scraped into the medium and pelleted, and episomal DNA was isolated by Hirt extraction (Hirt, B. 1967. Selective extraction of polyoma DNA from infected mouse cell cultures. J Mol Biol 26:365-9). The DNA was subjected to Southern analysis and hybridized with a biotinylated Pst I fragment of the pXX plasmid containing the Cap gene sequence. A DNA detection kit North2South™ Chemiluminescent Nucleic Acid Hybridization and Detection (PIERCE) was used to detect the AAV DNA after exposure to X-ray films.

Results

Design of a Dual Splicing Switch System using Lac-Z Gene as an Example

Previous efforts to make AAV packaging cell lines in 293 cells were focused on regulation of the expression of promoter p5 products Rep78 and Rep68. There has been success by substituting p5 with exogenous inducible promoters that partially resolve the problem of Rep-mediated toxicity. However, Rep 52/40 produced by promoter p19 could not be regulated and remained problematic in obtaining stable 293 cell-based packaging cell lines (Ogasawara et al. 1999); and Yang et al. 1994).

To simultaneously control the expression of all four AAV Rep proteins derived from both promoters p5 and p19, the coding sequences of all four Rep proteins were disrupted at their overlapping region, reasoning that this should effectively stop Rep gene expression altogether while keeping both promoters p5 and p19 intact and active. It is important to keep these two endogenous promoters intact, because they are proven to be the most effective for AAV vector production. However, after gene disruption the problem was created of how to restore the coding sequences when needed. A strategy was designed to achieve the above goal by taking advantage of an intron, three polyA sequences and DNA recombinase Cre and its recognition site LoxP.

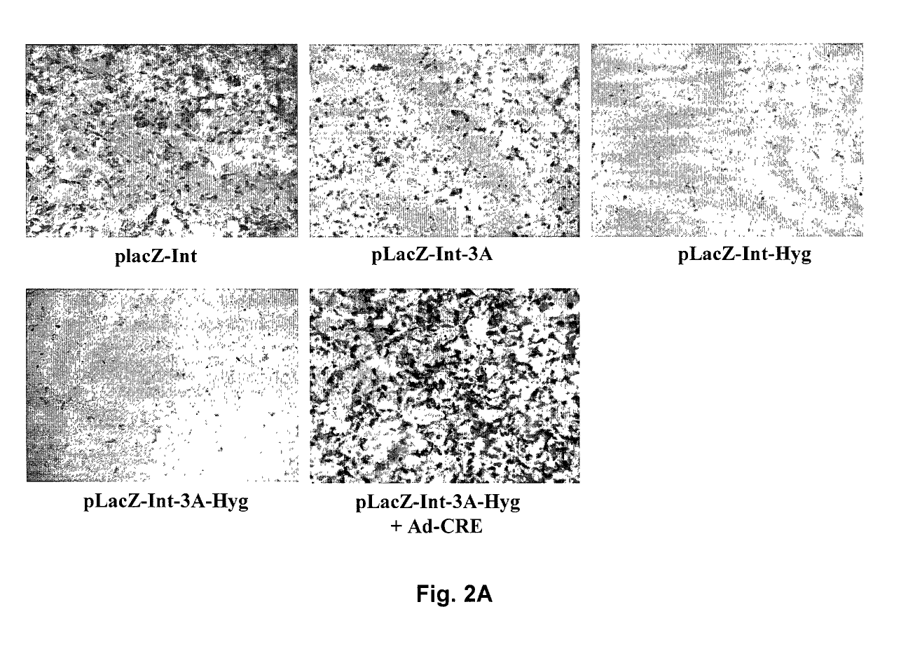

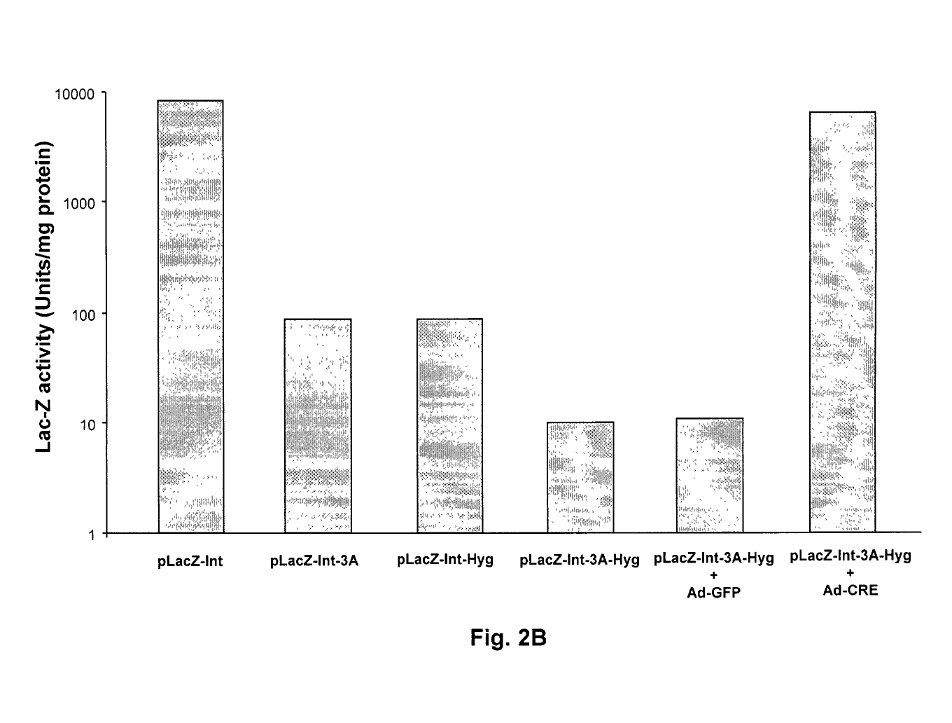

First, the P-galactosidase (Lac-Z) reporter gene was used as a model to test the dual splicing switch strategy. As illustrated in FIG. 1A, a small intron was inserted in the middle of the Lac-Z coding sequence (Sun et al. 2000). Subsequently, three SV40 polyA sequences in tandem were inserted in the middle of the intron. The insertion of three polyA signals would effectively terminate the transcription, yield a truncated mRNA, and render the gene nonfunctional. To restore the transcription of full-length mRNA, the polyA tandem must be removable. For this purpose, the Cre/LoxP system, where the site-specific recombinase Cre of bacteriophage P1 recognizes its target sequence LoxP sites and loops out the DNA sequences in between (Anton et al. 1995), was used. A LoxP site was placed both upstream and downstream to flank the polya tandem, generating plasmid pLacZ-Int-3A (FIG. 1A). As expected, this plasmid had much lower (˜80 fold) Lac-Z gene expression when compared to its parental plasmid pLacZ-Int (FIGS. 2A and 2B). However, low levels of leaky gene expression were still seen. This could be due to the very strong CMV promoter activity in 293 cells and the high copy numbers of plasmid transfected into the cells. It is also possible that the polyA tandem was not sufficient to completely terminate all transcription activity, perhaps because it was not long enough to allow for full termination. As a result, a 2.1 kb hygromycin-resistant gene cassette (including a TK-promoter and a polyA site) was further inserted downstream of the polyA tandem, generating plasmid pLacZ-Int-3A-Hyg (FIG. 1A). As a comparison, a similar plasmid pLacZ-Int-Hyg was constructed without the polyA tandem, but had the hygromycin-resistant gene cassette (FIG. 1A). Transfection of the above constructs in 293 cells demonstrated synergistic effects between the polyA tandem and hygromycin-resistant cassette in terminating Lac-Z gene transcription. The addition of the Hyg^{r} cassette in pLacZ-Int-3A plasmid further lowered the expression of the Lac-Z gene by approximately another 8 fold, a total of ˜640 fold decrease when compared to the original parental plasmid pLacZ-Int (FIG. 2B). The hygromycin-resistant cassette alone also decreased the Lac-Z gene expression by ˜80 fold in the pLacZ-Int-Hyg plasmid (FIG. 2B). In summary, insertion of the best transcription termination unit (three polyA sequences plus a Hyg^{r} cassette) diminished Lac-Z gene expression by as many as 640 fold (FIG. 2B).

The next step was to determine whether the gene expression from the gene containing the dual splicing switch could be induced back to its original levels upon delivery of the Cre gene by an adenovirus vector (Ad-Cre). Plasmid pLacZ-Int-3A-Hyg was transfected into 293 cells followed by Ad-Cre infection. X-gal staining of the cells after plasmid transfection and Ad-Cre infection showed dramatic induction of Lac-Z expression (FIG. 2A), and revealed no significant difference from its parental plasmid pLacZ-Int. Quantitative Lac-Z enzyme activity assay also showed consistent results (FIG. 2B). The Lac-Z activity from plasmid pLacZ-Int-3A-Hyg was induced by approximately 600 fold by Ad-Cre infection, but not by Ad-GFP control virus infection (FIG. 2B). In summary, the dual splicing switch system effectively stopped the gene expression from the middle of the coding sequence and gene expression could be effectively restored upon induction by Ad-Cre infection.

Tight Control of all Four Rep Proteins by the Dual Splicing Switch

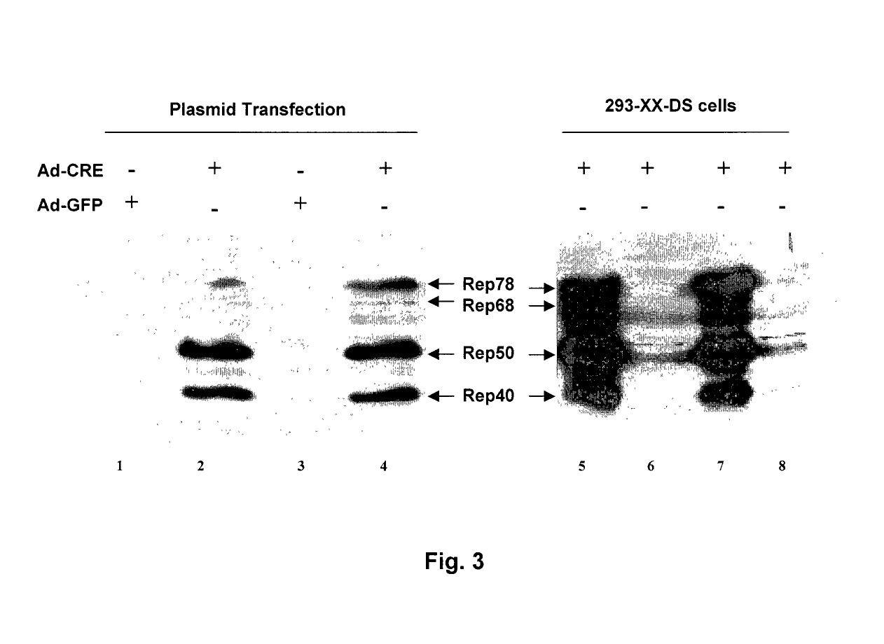

Equipped with the same strategy utilized in regulating the Lac-Z gene expression, the dual splicing switch (the Int-3pA-Hyg cassette) was inserted into the AAV Rep coding region downstream from the p19 promoter (FIG. 1B), disrupting all four Rep coding sequences. To compare the effect of different insertion loci, two sites in the shared Rep coding region were selected for insertion of the dual splicing switch. Two constructs, pXX-Int-3A-Hyg-2 and pXX-Int-3A-Hyg-8, were generated as described above. Western analysis of Rep protein expression showed similar results from both plasmids (FIG. 3, lanes 2 & 4). All four Rep proteins, particularly the two smaller Rep proteins, were induced from both plasmids after Ad-Cre infection. These two plasmids were further examined for their packaging function in producing an AAV-GPF vector (AAV-GPF vector was described in Xiao el al. 1998). Transient transfection experiments showed no difference in packaging functions between the two inducible plasmids and a commonly use AAV packaging plasmid pXX2 (Id.) (data not shown). These results were convincing, that gene expression of all four Rep proteins could be tightly controlled simultaneously and also be readily inducible using the dual splicing switch system.

To further investigate if the dual splicing switch could tightly control the AAV genes to generate stable 293 cell lines, plasmid pXX-Int-3A-Hyg-2 was transfected into 293 cells and selected for hygromycin-resistant colonies. Strikingly, a large number of drug resistant colonies formed after selection. This was in sharp contrast to a previous study using 293 cells (Yang el al. 1994), where only one clone named neo6 was identified containing Rep DNA. Even the establishment of neo6 cell was difficult at its initial stage due to a crisis period prior to the first 20 doublings (Id.). In that study only Rep78 and Rep68, but not Rep52 and Rep40, gene expression was regulated. By contrast, not only did the experiments described herein show a large number of healthy colonies formed, but also clones were identified having significant inducibility of Rep gene expression after Ad-Cre infection. Among numerous clones, two of them, (293-XX-DS-19 and 293-XX-DS-34) were identified as highly inducible for Rep expression (FIG. 3, lanes 5 & 7). Clone 293-XX-DS-19 was chosen as a packaging cell line precursor for the next step. Generation of stable and high-titer 293 cell-based AAV packaging cell lines

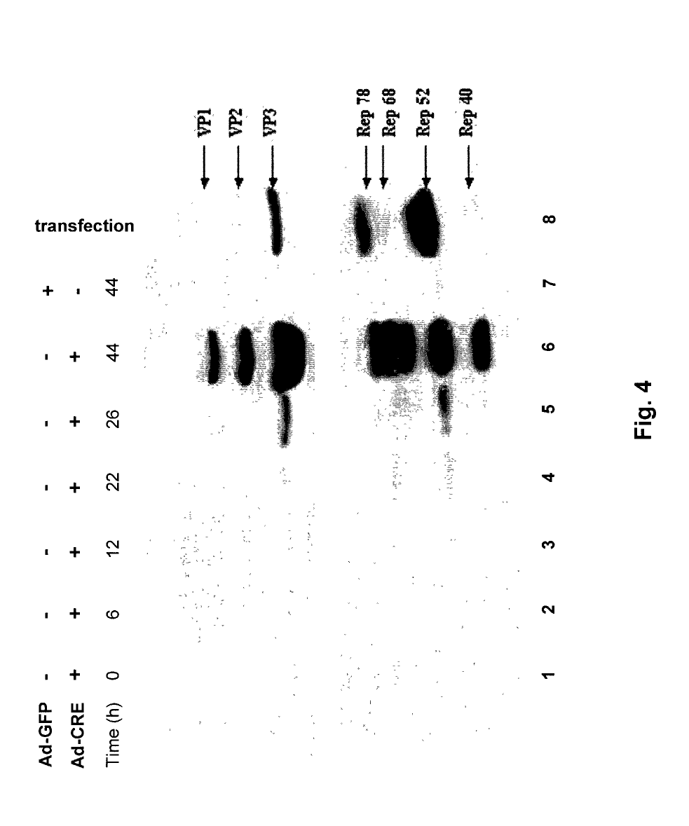

To establish an AAV packaging cell line, an AAV-GFP vector was delivered into the 293-XX-DS-19 cells as described above. To achieve a higher copy number of Rep and Cap genes in the cell line, the AAV-GFP vector sequence was delivered along with an additional copy of a packaging plasmid pXX-Int-3A-Hyg into the 293-XX-DS-19 cells. Thus, the AAV-GFP vector was first inserted into pXX-Int-3A-Hyg plasmid to generate pXX-Int-3A-Hyg-GFP (FIG. 1B). This plasmid also contained a Neo resistant gene as a marker for G418 selection. After transfection into 293-XX-DS-19 cells and G418 antibiotics selection, again a large number of drug-resistant colonies formed, suggesting that the Rep genes were tightly controlled and Rep-mediated toxicity was successfully averted. A time course of Rep and Cap gene expression after Ad-Cre helper virus infection was monitored at different intervals. The results showed that Rep and Cap expression started from 22 hours (FIG. 4, lane 4) and reached high-levels at 44 hours after Ad-Cre infection (FIG. 4, lane 6). As expected, AAV gene expression could not be induced by a control Ad vector, Ad-GFP, even after 44 hours of infection (FIG. 4, lane 7). Compared to the Ad-free transfection method (FIG. 4, lane 8), the cell line had significantly higher expression of capsid genes and Rep40 protein (FIG. 4, lane 6), which are particularly important for AAV vector packaging.

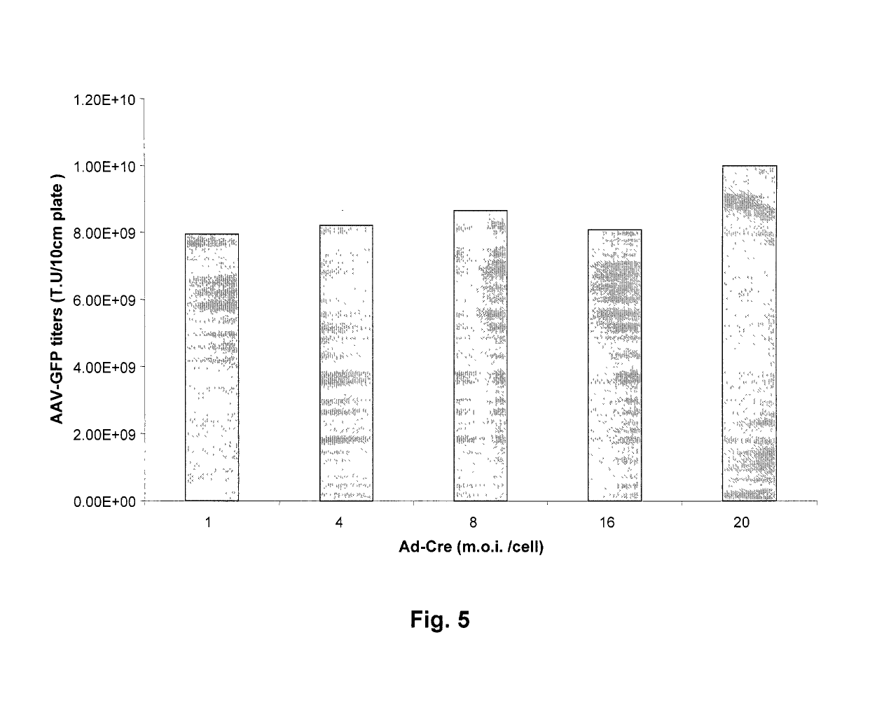

Among the 293 cell-based AAV-GFP cell lines, more than ten of them demonstrated vector yields greater than 10⁹ transducing units (t.u) per 10-cm plate. One cell line, 293-GFP-145, reached as high as 8.6×10⁹ t.u./10-cm plate. Optimization of helper Ad-Cre multiplicity of infection (m.o.i) ranging from 1 to 20 showed little improvement in AAV vector yields from 293-GFP-145 cells (FIG. 5), suggesting a broad range of tolerance for Ad-Cre helper function. Furthermore, the AAV packaging cell lines created by the dual splicing strategy have been very stable. For example, the 293-GFP-145 cell line has been cultured continuously for more than six months. The growth rate was very similar to the parental 293 cells. Importantly, the capacity of producing AAV-GFP vector remained the same after consecutive passages (data not shown).

To demonstrate that the success in AAV-GFP cell lines can be reproduced in other cell lines carrying AAV vectors of different transgenes, a similar strategy was used to establish another packaging cell line containing a human dystrophin minigene (Wang el al. 2000). Because there is no drug resistant marker gene in the AAV-mini-dystrophin vector, the packaging plasmid pXX-Int-3A-Puro (FIG. 1B) was co-transfected into the 293-XX-DS-19 cells. Plasmid pXX-Int-3A-Puro is identical to plasmid pXX-Int-3A-Hyg except that the hygromycin gene was replaced by the puromycin resistant gene for selection (de la Luna, S., Soria, I., Pulido, D., Ortin, J. and Jimenez. A. 1988. Efficient transformation of mammalian cells with constructs containing a puromycin-resistance marker. Gene 62:121-6). Reproducibly, a large number of colonies formed after puromycin selection of the AAV-mini-dystrophin vector packaging cell lines. Numerous clones were isolated and challenged with Ad-Cre helper virus and then monitored for vector production by DNA dot blot method. Again, more than 10 clones yielded AAV vectors greater than 10¹² viral particles (v.g) per 10-cm plate (data not shown). Some cell clones were further subcloned and expanded for larger scale AAV vector production. Satisfactory vector yields were obtained from those packaging cell lines. For example, large scale vector preparation from the AAV-GPF producer cell line 293-GFP-145 (20×15-cm plates) generated 6.8×10¹³ vector genome particles after heparin affinity column chromatography purification (Zolotukhin el al. 1999). The AAV-mini-dystrophin vector cell line 293-AAV-3999-70 (20×15-cm plates) generated 1.24×10¹⁴ vector genome particles after column purification.

Characterization of AAV Vector Produced in the 293-GFP-145 Cells

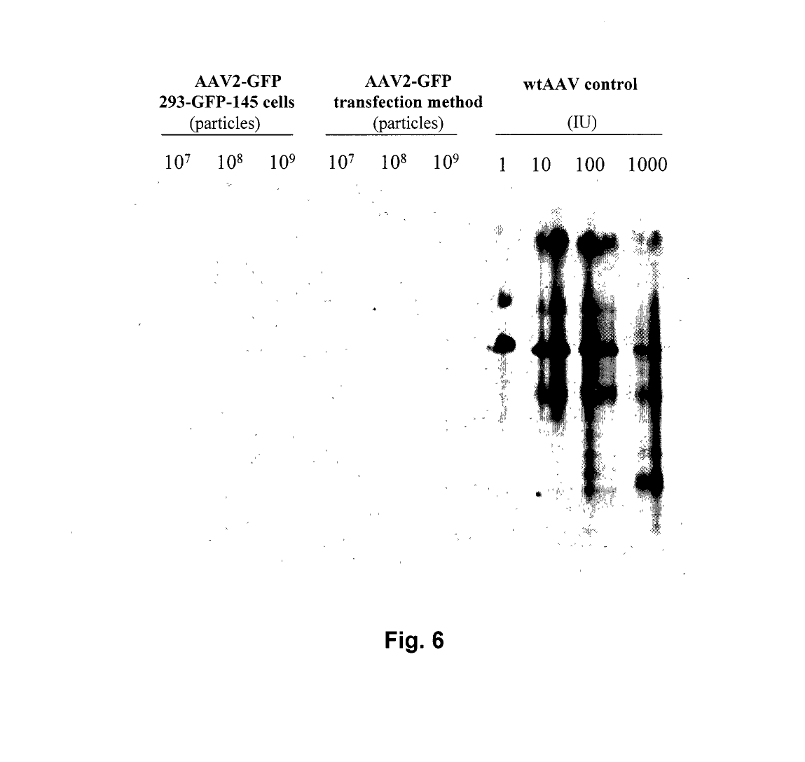

A clinically useful cell line should produce vectors free of replication-competent AAV (rcAAV, also termed wild-type-like AAV). Therefore, the 293-GFP-145 cell line was examined to determine if that cell line could generate rcAAV, which would most probably be derived from non-homologous recombination between the vector and packaging sequences during AAV production. An infection-based viral amplification assay was selected to detect the rcAAV, because this method is highly sensitive. Up to 10⁹ viral genome particles of highly purified AAV-GFP, generated either from the 293-GFP-145 cells or from the triple plasmid transfection method, were used to infect 293 cells, which were co-infected with wild-type adenovirus to provide helper functions for rcAAV propagation. As a positive control, wild-type AAV also was used similarly to infect the 293 cells at multiplicities ranging from 1 to 1000 infectious units along with wild-type adenovirus co-infection. The viruses were harvested following full cytopathic effect and amplified again by infecting fresh 293 cells. Southern analysis of the viral DNA isolated after the second amplification revealed no detectable AAV coding sequences from the two individual AAV-GFP vector stocks (FIG. 6). However, strong signals of AAV monomer and dimer replication intermediates were detected in the wild-type AAV positive control samples at all m.o.i.s from 1 to 1000 (FIG. 6). These results indicated that there is no detectable rcAAV in up to 10⁹ AAV-GFP viral genome particles produced either by the 293-GFP-145 cell line or by the triple plasmid transfection method (Xiao et al 1998).

The ratios of viral genome particles versus the transducing units (v.g./t.u) were then compared, which is an indicator of AAV vector infectivity. In these experiments, AAV-GFP viruses, derived from an identical vector backbone but produced by three different production methods, were subjected to the comparison. When the vector titers were measured by transducing units (each t.u. generates one GFP-positive green cell after infection), packaging cell line 293-GFP-145 produced the highest titer (>8×10⁹ t.u./10-cm plate), followed by triple-plasmid transfection (Xiao et al 1998) (1.5×10⁹ t.u./10-cm plate), and then a HeLa cell-based packaging cell line XX-GFP-53 (1×10⁹ t.u./10-cm plate. Moreover, the AAV-GFP vector v.g./t.u. ratios of the above three methods were respectively 80, 500, and 2000 for the 293-GFP-145 cell line, triple-transfection, and the XX-GFP-53 cell line, respectively, suggesting that AAV vector generated by the 293-GFP-145 cell line had the highest infectivity.

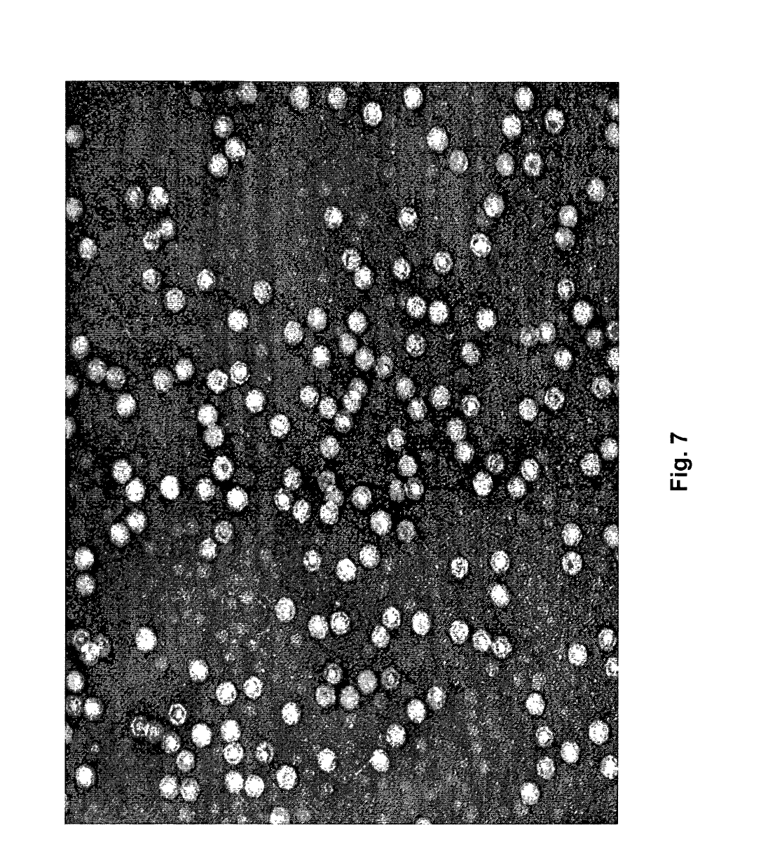

Transmission electron microscopy was used to study the morphology of the AAV virions produced from the 293-GFP-145 cells (FIG. 7). The viral particles were purified by heparin affinity column chromatography method without separation of empty particles from full particles by density gradient centrifugation. Quantitation of a large number of virions revealed that approximately one-half of the virions were dense full particles while the other half were empty or defective particles having an electron-lucent inner sphere.

Molecular Characterization of 293-Based AAV Packaging Cell Lines

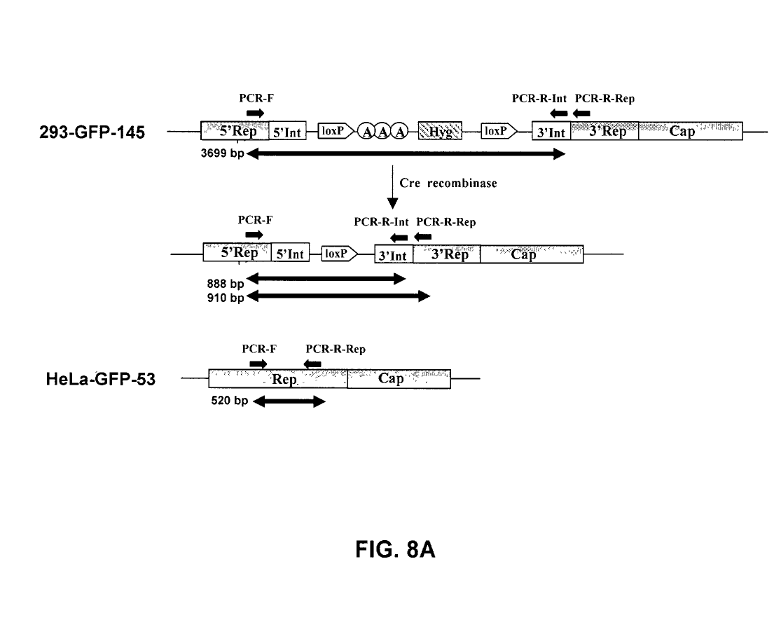

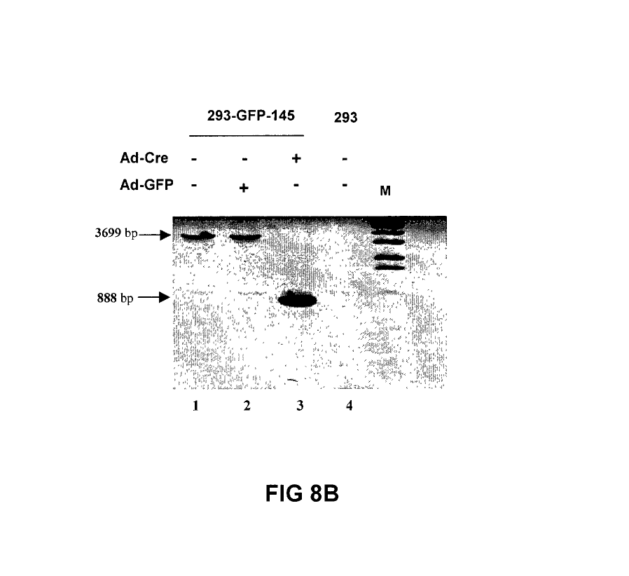

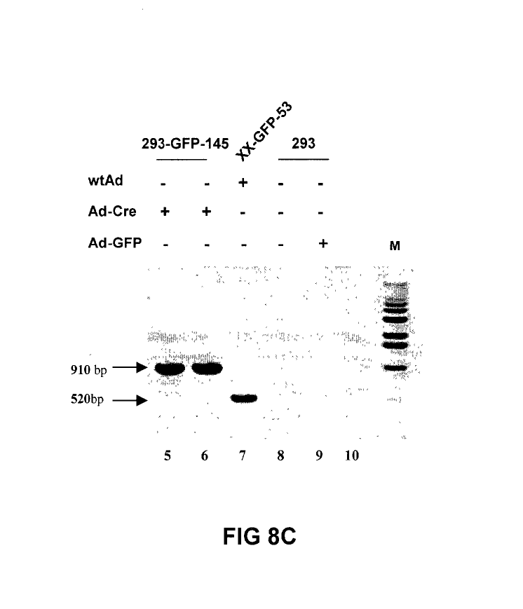

Because the induction of AAV Rep genes is accomplished by the removal of the transcription termination cassette (three polyA signals plus a drug resistant gene) by the Cre enzyme expressed from the Ad-Cre vector, the efficiency of the Ad-Cre infection in excising of the termination cassette in the Rep coding sequence was studied. PCR primers flanking the termination cassette were designed to detect the DNA before and after deletion of the Int-3A-Hyg cassette (FIG. 8A) in 293-GFP-145 cells. The forward primer (PCR-F) was in the Rep gene while the reverse primer (PCR-R-Int) was in the 3′ region of the inserted intron (FIG. 8A). DNA isolated from 293-GFP-145 cells before Ad-Cre infection revealed a 3699 bp PCR product (FIG. 8A, and 8B lane 1). Control Ad-GFP virus infection did not result in any deletion and therefore yielded the same 3699 bp PCR product (FIG. 8B, lane 2). As expected, Ad-Cre infection of this cell line yielded an 888 bp PCR product as a result of the deletion of the 2.8 kb termination cassette by the Cre enzyme (FIG. 8B, lane 3). In addition, the Cre-mediated excision was so efficient that no parental DNA (3699 bp) could be detected (FIG. 8B, lane 3). To further confirm the absence of wild-type Rep sequence in the 293-GFP-145 cell line, another reverse primer (PCR-R-Rep) located in the 3′ Rep gene was paired with the forward primer (PCR-F) located in the 5′ Rep sequence, flanking the artificial intron insertion (FIG. 8A). Wild-type Rep coding sequence from HeLa cell-based cell line XX-GFP-53 generated a 520 bp PCR product (FIG. 8C, lane 7), while DNA isolated from Ad-Cre infected 293-GFP-145 cell generated a 910 bp PCR product (FIG. 8C, lanes 5 & 6), which was the sum of the 520 bp wild-type Rep sequence plus the artificial intron (338 bp) and the remaining LoxP site (52 bp) post-excision of the termination cassette. Finally, PCR products from low-molecular-weight DNA (Hirt extraction, FIG. 8C, lane 5) as well as total cellular DNA (FIG. 8C, lane 6) reveal the same intensity, an indication of episomal amplification of AAV coding sequence (see below for Southern analysis results).

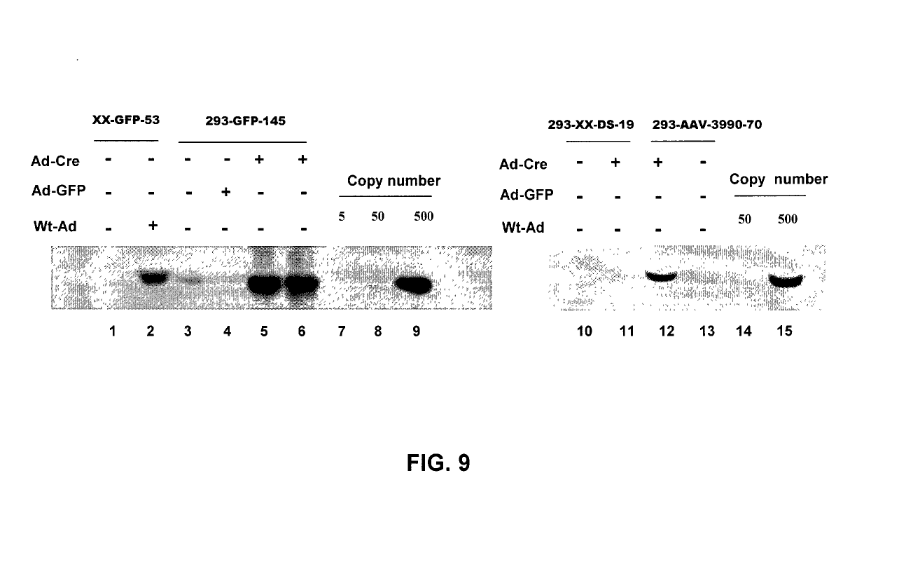

Because selective Rep-Cap gene amplification is a mechanism for high-titer AAV production from HeLa cell-based stable cell lines, it was then examined whether this phenomenon is also responsible for the high-titer vector production in the 293 based producer cell lines. Southern analysis was performed on both total DNA and episomal DNA (Hirt extraction) to see whether the Rep-Cap genes were amplified during vector production. As shown in FIG. 9, the HeLa cell-based XX-GFP-53 cell line had its Rep-Cap genes amplified from four copies per cell (FIG. 9, lane 1 and Li et al., unpublished results) to about 200 copies (FIG. 9, lane 2). Similarly, the 293-GFP-145 cell line had its Rep-Cap genes amplified from about 50 copies (FIG. 9, lane 3) to more than 500 copies per cell after Ad-Cre infection in both fractions of total DNA (FIG. 9, lane 5) and episomal DNA (FIG. 9, lane 6). This amplification was Rep-dependent because Ad-GFP infection in the 293-GFP-145 cell could not result in any amplification of the AAV genes (FIG. 9, Lane 4). Amplification of Rep-Cap genes also was observed in the mini-dystrophin producer cell line 293-AAV-3A990-70. The initial Rep-Cap copy number in this cell line also was around 50 (FIG. 9, lane 13), and was amplified to about 250 copies after Ad-Cre infection (FIG. 9, lane 12).

Aiming at simultaneous and tight control of all four Rep proteins' gene expression, a dual splicing strategy was devised that blocked the gene expression in the middle of the protein coding sequence. Because the four Rep proteins share the majority of their coding sequences and the promoter p19 is located in the middle of the Rep78 and Rep68 coding sequences, it was reasoned that disruption of the Rep genes downstream of promoter p19 should stop expression of all four Rep proteins. An intron was inserted into the shared coding sequence of the four Rep proteins to disrupt the gene (FIG. 1B). The advantage of inserting an intron is that theoretically it can be inserted anywhere in the transcribed region, coding or noncoding, and also can be readily and accurately removed through RNA splicing. As a result, the risk of introducing mutations into the coding sequence is minimized. It has been shown previously that introns can tolerate insertions (Sun el al. 2000).

As shown herein, the insertion of polyA sequences into the intron effectively terminated transcription and decreased gene expression by 80 fold. Moreover, the addition of a drug resistant gene cassette further decreased the gene expression by another 8 fold for a total of over 640 fold diminution. Two LoxP sites flanking the transcription termination cassette (polyA sites plus the Hyg^{r} gene) alleviated the transcription blockade by Cre recombinase-mediated DNA splicing, resulting in effective restoration of full-length transcription and gene expression.

A significant advantage of the terminating intron system to control AAV Rep gene expression is that it permitted simultaneous control of expression of all four Rep protein genes. This was not the case in previous efforts by others in making 293 cell-based AAV packaging cell lines. Merely controlling promoter p5 products could not eliminate the cytostatic and cytotoxic effects from leaky expression of p19 products Rep52 and Rep40 (Chadeuf et al. 2000; Ogasawara et al. 1999; Okada et al. 2001 and Yang et al. 1994). Secondly, very tight control of Rep gene expression also is important. As shown herein, besides the triple polyA sites, a drug-resistant gene as part of the termination cassette also was used. It not only rendered synergistic effects in terminating the Rep gene transcription with the three polyA sites, but also served as a selectable marker for stable cell line establishment.

The stringency of Rep gene expression control in this system was partially reflected by the ease of obtaining stable cell clones after transfection and antibiotics selection. Hundreds of drug-resistant colonies were consistently obtained during the process of cell line development. Most of the clones exhibited a normal growth rate when compared to the parental 293 cells. A large majority of those cell clones retained their inducibility of Rep gene expression after Ad-Cre infection, suggesting that the Rep gene under the control of the dual splicing switch was very tight and nontoxic. For example, cell line 293-XX-DS-19 harbors approximately 50 copies of the dual splicing switch-controlled AAV Rep-Cap genes. Nonetheless, no toxicity was observed and the cell line has been very stable throughout the long-term passage and subcloning process. Its progeny cell lines also exhibited similar stability after obtaining additional copies of the controlled Rep-Cap genes along with the AAV vector sequences, that is, AAV-GFP and AAV-mini-dystrophin vectors.

Notably, AAV Rep and Cap gene amplification occurred in the GFP and mini-dystrophin cell lines by 5 to 10 fold during vector production. The amplification phenomenon has been observed in a number of HeLa cell-based AAV packaging cell lines, which showed high titer AAV vector production (Chadeuf et al. 2000; Liu, X., Voulgaropoulou, F., Chen, R., Johnson, P. R. and Clark, K. R. 2000. Selective Rep-Cap gene amplification as a mechanism for high-titer recombinant AAV production from stable cell lines. Mol Ther 2:394-403; Nony, P., Tessier, J., Chadeuf, G., Ward, P., Giraud, A., Dugast, M., Linden, R. M., Moullier, P. and Salvetti, A. 2001. Novel cis-acting replication element in the adeno-associated virus type 2 genome is involved in amplification of integrated rep-cap sequences. J Virol 75:9991-4; and Tessier et al. 2001). In the 293 cell-based cell lines, the cells already had approximately 50 copies of the Rep and Cap gene. Further amplification of these genes is believed to further boost AAV vector titers.

An additional advantage of the terminating intron control in the AAV embodiment is the preservation of the AAV endogenous promoters (p5 and p19), which have proven to be the best regulatory elements for ensuring optimal viral gene expression both temporally and quantitatively during AAV vector production (Pereira, D. J., McCarty, D. M. and Muzyczka, N. 1997. The adeno-associated virus (AAV) Rep protein acts as both a repressor and an activator to regulate AAV transcription during a productive infection. J Virol 71:1079-88; and Xiao et al. 1998). A very important distinction between the strategy described herein and other strategies is that in the present system, there is no selection pressure to inactivate both p5 and p19 promoters in order to obtain a packaging cell line. By contrast, p5 and p19 promoter shut-off is a prerequisite in previous studies, where these two promoters were expected to turn back on in the packaging cell lines after adenovirus infection. However, long-term promoter shut-off is believed to lead to DNA methylation which makes re-activation difficult. This phenomenon seems true in previous 293 cell-based cell lines, especially for promoter p 19 (Ogasawara et al. 1999; Okada et al. 2001 and Yang et al 1994). This phenomenon also may explain in part why some HeLa cell-based AAV packaging cell lines required very high doses of wild-type adenovirus infection to re-activate AAV gene expression (Liu et al. 2000). In the 293 cell-based cell lines described herein, however, promoters p5 and p19 are expected to remain constitutively active without causing a deleterious effect to the cells, because the mRNAs were terminated prematurely and rendered non-functional until Ad-Cre infection, which restores the coding regions. Thus, the promoters maintain their activities and their responsiveness to Ad helper infection at all times. This is believed to be a contributing factor for the modest dose requirement of Ad-Cre infection and the resulting high-titer AAV vector production in the cell lines described herein. Continuity of promoter expression and the benefits thereof, despite lack of expression of the target gene, is characteristic of any target gene controlled by the dual splicing switch.

Having succeeded in controlling both Lac-Z and AAV Rep gene expression, this technology also should be useful for a large variety of other applications. For example, to control genes containing multiple overlapping open reading frames found in other viruses, and for genes that require regulation by their own endogenous promoters in animal and plant models. Non-limiting examples of viruses suitable for these purposes include members of the Family Parvoviridae, having non-structural proteins (NS proteins), such as, without limitation, autonomous parvoviruses including B 19 and minute mice virus.

Transgenic Mice Exhibiting Tissue-Specific Expressions of LacZ

Transgenic mice are generated by blastocyst injection, but may be generated by any suitable method, many of which are well known and are very effective. Mice are prepared having a LacZ gene interrupted by a dual splicing switch (DSS) as described herein (pLacZ-Int-3A-Hyg). In one case, the LacZ-DSS⁺ mice are injected with a Cre-tat fusion protein (Peitz et al. 2002), which should result in expression of LacZ in most tissues. In an alternate experiment, transgenic derivatives of the LacZ-DSS⁺ mice are derived having the Cre protein expressed under MCK (muscle-specific creatine kinase) promoter control. The LacZDSS gene is introduced into a mouse embryo on the same nucleic acid (plasmid) as the MCK-Cre gene. LacZ and GFP expression is expected in muscle tissue, but not in other tissues.

I claim:

1. An isolated and purified nucleic acid comprising an intron comprising a disruptable transcription termination sequence.

2. The nucleic acid of claim 1, wherein said intron is positioned in an expressed sequence of a target gene.

3. The nucleic acid of claim 2, wherein said target gene is an AAV Rep gene.

4. The nucleic acid of claim 3, wherein said intron is inserted between P19 and P40 promoters of said Rep gene.

5. The nucleic acid of claim 4, wherein said nucleic acid further comprises an AAV Cap gene.

6. The nucleic acid of claim 2, wherein said intron is positioned within an overlapping reading frame of a target gene comprising two or more overlapping reading frames.

7. The nucleic acid of claim 1, wherein said disruptable transcription termination sequence is excisable.

8. The nucleic acid of claim 7, wherein said excisable transcription termination sequence comprises one of a polyA sequence and a gene.

9. The nucleic acid of claim 7, wherein said excisable transcription termination sequence comprises a polyA signal and a gene.

10. The nucleic acid of claim 9, wherein said gene is a selectable marker.

11. The nucleic acid of claim 7, wherein said intron comprises a pair of recombination sites in the same orientation flanking said excisable termination sequence, such that said excisable transcription termination sequence can be excised by a recombinase enzyme.

12. The nucleic acid of claim 11, wherein said recombination sites are LoxP sites or a derivative, analog or homologue thereof.

13. The nucleic acid of claim 7, wherein said excisable transcription termination sequence comprises a polyA sequence and a second gene flanked by a pair of LoxP sites or derivatives, analogs or homologues thereof.

14. The nucleic acid of claim 13, wherein said excisable transcription termination sequence comprises three polyA sequences 5′ to said second gene.

15. The nucleic acid of claim 1, wherein said disruptable transcription termination sequence comprises one of a polyA sequence and a second gene.

16. The nucleic acid of claim 15, wherein said disruptable transcription termination sequence comprises one of a polyA sequence and a second gene.

17. The nucleic acid of claim 2, wherein said target gene is parvovirus non-structural gene and said intron is inserted in a position in said non-structural gene to substantially inhibit expression of all reading frames of said non-structural gene.

18. A cell or cell line comprising a nucleic acid comprising a gene having an intron comprising a disruptable transcription termination sequence.

19. The cell or cell line of claim 18, wherein said intron is positioned in an expressed sequence of a target gene.

20. The cell or cell line of claim 19, wherein said target gene is a parvovirus non-structural gene and the intron is positioned within an overlapping reading frame of the non-structural gene.

21. The cell or cell line of claim 20, wherein said non-structural gene is an AAV Rep gene.

22. The cell or cell line of claim 21, wherein said cell or cell line comprises an AAV Cap gene.

23. The cell or cell line of claim 21, further comprising a nucleic acid template for a packageable rAAV genome.

24. The cell or cell line of claim 23, wherein said packageable rAAV genome comprises a dystrophin gene.

25. The cell or cell line of claim 21, wherein said cell or cell line is derived from 293 cells.

26. The cell or cell line of claim 19, wherein said target gene is an AAV Rep gene and said intron comprises, in a 5′ to 3′ direction, a first LoxP site, or a derivative analog or homologue thereof, three polyA sequences, a gene for a selectable marker, and a second LoxP site or a derivative analog or homologue thereof, wherein said intron is positioned in a coding sequence shared by Rep78, Rep68, Rep52 and Rep40 proteins and said cell or cell line further comprises a nucleic acid template for a packageable rAAV genome.

27. The cell or cell line of claim 25, wherein the intron is positioned between the P19 and P40 promoters of the Rep gene.

28. A transgenic non-human animal comprising a nucleic acid comprising a target gene having an intron positioned in an expressed sequence of the target gene, the intron comprising a disruptable transcription termination sequence.

29. A method for expressing a gene in a cell, comprising the step of contacting a nucleic acid in a cell comprising an inactivated target gene having an intron comprising an disruptable transcription termination sequence with a trans-acting agent for disrupting the transcription termination sequence.

30. The method of claim 29, further comprising, before the contacting step, the step of transferring into the cell the inactivated target gene.

31. The method of claim 29, wherein the contacting step includes the step of transferring into the cell a trans-acting agent to disrupt the disruptable transcription termination sequence.

32. The method of claim 31, wherein the trans-acting agent is transferred into the cell by transfer of a gene for expressing the trans-acting agent.

33. The method of claim 32, wherein the gene is transferred by a recombinant adenovirus.

34. The method of claim 29, wherein the disruptable transcription termination sequence includes at least one of a polydenylation sequence and a gene.

35. The method of claim 29, wherein the transcription termination sequence is flanked by recombination sites and the trans-acting agent is a recombinase.

36. The method of claim 35, wherein the recombination sites are LoxP sites and the recombinase is Cre.

37. The method of claim 35, wherein the second gene is a selectable marker gene.

38. The method of claim 35, wherein the recombinase is introduced into the cell or cell line by protein transfer or by a gene for expressing the recombinase in the cell.

39. A method for producing a recombinant AAV particle, comprising the step of introducing a recombinase enzyme into a cell containing:
(a) a first nucleic acid sequence comprising AAV sequences encoding AAV Rep and Cap genes, wherein the Rep gene contains an intron positioned in a coding sequence shared by Rep78, Rep68, Rep52 and Rep40 proteins, the intron comprising a transcription termination sequence flanked by a pair of recombination sites; and
(b) a template for a packageable rAAV genome.

40. A method for producing recombinant parvovirus particles, comprising the step of introducing a recombinase enzyme into a cell containing:
(a) a first nucleic acid sequence comprising sequences for a parvovirus non-structural gene encoding two or more proteins from a shared reading frame, and any additional parvovirus proteins necessary in trans for production of recombinant parvovirus transducing unit, wherein the non-structural gene contains an intron positioned in the shared reading frame of the non-structural gene, the intron comprising a transcription termination sequence flanked by a pair of recombination sites; and
(b) a template for a packageable recombinant parvovirus genome.

41. An isolated and purified nucleic acid comprising an intron comprising a disruptable transcription termination sequence.

42. A cell or cell line comprising a nucleic acid comprising a gene having an intron comprising an excisable transcription termination sequence.

43. A method for expressing a gene in a cell, comprising the step of contacting a nucleic acid in a cell comprising an inactivated target gene having an intron comprising an excisable transcription termination sequence with a trans-acting agent for excising the transcription termination sequence.


SEQUENCE LISTING (7 sequences)

SEQ ID NO: 1 - DNA, 52 bp, Bacteriophage P1
cgggatccataacttcgtataatgtatgctatacgaagttatccagatcttc

SEQ ID NO: 2 - DNA, 1553 bp, Bacteriophage P1
tgcgcagctggacgtaaactcctcttcagacctaataacttcgtatagcatacattatacgaagttatattaagggttattgaatatgatcaatttacctgtaaatccatacagttcaataccttagcaggtcaaatagtgaccacttgatcatttgatcaaggttgcgctacgtaaaatctgtgaaaaattggcggtgttagtcctacagatttcgcgtaccacttagcaccaccaatcaatcagaggtgaaaaatgggatattcaactgctaaagtgtccactcatcttgagcttgagaaaaaccgtggttactggcgggcaaaagggtttgatcgtgatagttgccaactgtcattatcgcgcggtgaagagaaaatagaacgcacgcgcggtcgctggcgtttctatgacgagaaccataaacaggtaaaggcagagccgatcctgtacactttacttaaaaccattatctgagtgttaaatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagccattaacgcgtaaatgattgctataattagttgata

SEQ ID NO: 3 - DNA, 48 bp, Saccharomyces cerevisiae
gaagttcctatactttctagagaataggaacttcggaataggaacttc

SEQ ID NO: 4 - DNA, 1272 bp, Saccharomyces cerevisiae
atgccacaatttggtatattatgtaaaacaccacctaaggtgcttgttcgtcagtttgtggaaaggtttgaaagaccttcaggtgagaaaatagcattatgtgctgctgaactaacctatttatgttggatgattacacataacggaacagcaatcaagagagccacattcatgagctataatactatcataagcaattcgctgagtttcgatattgtcaataaatcactccagtttaaatacaagacgcaaaaagcaacaattctggaagcctcattaaagaaattgattcctgcttgggaatttacaattattccttactatggacaaaaacatcaatctgatatcactgatattgtaagtagtttgcaattacagttcgaatcatcggaagaagcagataagggaaatagccacagtaaaaaaatgcttaaagcacttctaagtgagggtgaaagcatctgggagatcactgagaaaatactaaattcgtttgagtatacttcgagatttacaaaaacaaaaactttataccaattcctcttcctagctactttcatcaattgtggaagattcagcgatattaagaacgttgatccgaaatcatttaaattagtccaaaataagtatctgggagtaataatccagtgtttagtgacagagacaaagacaagcgttagtaggcacatatacttctttagcgcaaggggtaggatcgatccacttgtatatttggatgaatttttgaggaattctgaaccagtcctaaaacgagtaaataggaccggcaattcttcaagcaataaacaggaataccaattattaaaagataacttagtcagatcgtacaataaagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataa

SEQ ID NO: 5 - protein, 11 aa, Human immunodeficiency virus
YGRKKRRQRRR

SEQ ID NO: 6 - DNA, 18 bp, Artificial Sequence
  HCG-Int forward PCR primer
gtaagaagatccgaggtc

SEQ ID NO: 7 - DNA, 21 bp, Artificial Sequence
  HCG-Int reverse PCR primer
ccttgtccggttaccctgcag